SEQUENCE LISTING (2 sequences)

SEQ ID NO: 1 - DNA, 25 bp, Artificial Sequence
  Probe for FOXO1
tcccccagccgcaggagagccaaga

SEQ ID NO: 2 - DNA, 25 bp, Artificial Sequence
  Probe for ETV6
catgtctcacagcgagagagatcag

(19) United States
(12) Patent Application Publication
(10) Pub. No.: US 2021/0292765 A1
(43) Pub. Date: Sep. 23, 2021

(54) COMPOSITIONS COMPRISING INTERMEDIATE NON-CODING RNA REGULATORS MODULATING THE EXPRESSION OF ETV6 OR FOXO1 AND USES THEREOF

(71) Applicant: University of Exeter, Exeter Devon (GB)

(72) Inventors: Lorna Harries, Exeter Devon (GB); Eva Latorre, Exeter Devon (GB)

(21) Appl. No.: 17/263,465

(22) PCT Filed: Jul. 29, 2019
(86) PCT No.: PCT/GB2019/052125

§ 371 Date: Jan. 26, 2021

(30) Foreign Application Priority Data
Jul. 27, 2018 (GB) 1812334.9

(51) Int. Cl. C12N 15/113; A61P 39/00; A61P 35/00

(52) U.S. Cl. CPC C12N 15/1135; A61P 39/00; A61P 35/00; C12N 2310/113; C12N 2310/141; C12N 2320/31; C12N 15/113

(57) ABSTRACT

The present invention relates to: compositions for modulating the expression of FOXO1 and/or ETV6, the composition comprising one or more intermediate non-coding RNA regulators; attenuating splicing factor expression, the composition comprising an expression modulator of FOXO1 and/or ETV6; attenuating cell senescence and/or re-entry to cell cycle, the composition comprising an expression modulator of FOXO1 and/or ETV6 or their downstream targets related to splicing factor expression; or a composition capable of modulating splicing factor expression, the composition comprising one or more compounds able to bind to, or inhibit, FOXO1 and/or ETV6 genes or their downstream targets related to splicing factor expression. Such compositions have therapeutic benefits in the prevention, management, amelioration or treatment of an age-related disease or condition or cancer or also as a research tool/reagent.

### TECHNICAL FIELD OF THE INVENTION

The present invention relates to compositions which modulate expression of FOXO1 and/or ETV6 or their targets pertaining to the regulation of splicing factors. Such compositions have therapeutic potential in the prevention, management, amelioration or treatment of an age-related disease or condition or cancer and also as a research tool/reagent.

### BACKGROUND TO THE INVENTION

Senescent cells are viable and metabolically active entities that over multiple rounds of cell division have lost the ability to proliferate and have been shown to accumulate during the ageing process in multiple tissues and in multiple species (Faragher et al. 2017). Senescent cells release a cocktail of pro-inflammatory cytokines and remodelling proteins termed the senescence-associated secretory phenotype (SASP), which triggers the establishment of senescence in neighbouring cells in a paracrine manner and acts to further stimulate inflammation in surrounding tissues (Salama et al. 2014). Mounting evidence suggests that the increased senescent cell load contributes directly to organismal ageing (de Magalhaes 2004) and age-related disease (van Deursen 2014); targeted depletion of senescent cells in transgenic mice improves multiple ageing phenotypes and extends lifespan (Baker et al. 2016; Baar et al. 2017). Understanding the fundamental biology of cellular senescence, and importantly, the factors that contribute to it, is thus of key importance.

One area that is emerging as a potential driver of cellular senescence and ageing phenotypes is dysregulation of alternative splicing (Deschenes & Chabot 2017; Latorre & Harries 2017). Fine control of gene expression is essential for control of cellular function, plasticity and cellular identity. Changes in the expression of the regulatory machinery that govern splice site choice are seen in ageing human populations (Harries et al. 2011), in aged cells of multiple lineages (Holly et al. 2013), and are also linked with lifespan in animal models (Heintz et al. 2016; Lee et al. 2016). Age-related diseases as Alzheimer's disease, Parkinson's disease or cancer are also characterised by large-scale dysregulation of splicing, highlighting the importance of correct splicing for health throughout the life course (Latorre & Harries 2017). Splicing factors are good candidates for target genes to influence cell senescence, since several are tightly linked with control of proliferation, and some have roles in maintenance of telomere function (Kang et al. 2009; Anczukow et al. 2012). Loss of regulated alternative splicing in ageing tissues may therefore underpin the deterioration in response to intrinsic and extrinsic cellular stressors that characterises ageing in multiple species (Kourtis & Tavernarakis 2011) and has potential to be a major contributors to increased physiological frailty.

The upstream drivers of age-related dysregulation of splicing remain to be determined. Genes encoding splicing factors are themselves regulated by alternative splicing, and this unsurprisingly represents a strong contributor to their expression (Lareau & Brenner 2015). Regulation of the activity of splicing factors at the protein level is also known to be determined by the action of SRPK protein kinases, and also by PI3K/PTEN/AKT signalling at the level of phosphorylation and subcellular localisation (Blaustein et al. 2005; Bullock & Oltean 2017). Previous studies have suggested that some splicing factors may be regulated by alterations in RAF/MEK/ERK signalling (Tarn 2007). The concept of dysregulated cellular signalling during ageing is not a new one. The role of insulin/insulin-like growth factor 1 (IGF1/INS) signalling in ageing is well known and represents the first molecular pathway to be linked to ageing (Cohen & Dillin 2008); many genetic mutations within this pathway have been shown to extend lifespan (Suh et al. 2008). Manipulation of the IGF-1/INS pathway by genetic modification or dietary restriction have also demonstrated the importance of these pathways in extension of human lifespan (van Heemst et al. 2005) and has also been associated with longevity in model systems (Slack et al. 2015). RAF/MEK/ERK and PI3K/PTEN/AKT signalling intersect just downstream of IGF-1/INS signalling, and are also activated by classical ‘ageing’ stimuli such as DNA damage, dysregulated growth factors and inflammation (Fontana et al. 2012; Lin et al. 2013).

Some studies have suggested that the use of MEK or PI3K inhibitors could prevent the induction of cellular senescence and ageing (Demidenko et al. 2009; Chappell et al. 2011). The NFκB pathway, which regulates the senescence-associated secretory phenotype (SASP) (Salminen et al. 2012), is also known to intersect with ERK and AKT signalling (Lin et al. 2012), suggesting that inflammatory changes could lie both upstream and downstream of RAF/MEK/ERK signalling. The relationship between these pathways is however not straightforward; there is crosstalk between them and also effects of dose, cell type and context (Rhim et al. 2016). Activation of ERK and AKT signalling by classical ageing stimuli such as DNA damage, inflammation or growth factors may therefore induce dysregulation of splicing factor expression and alternative splicing and influence cellular senescence.

It is an object of the present invention to provide a composition which is capable of one or more of the following: attenuating gene expression of FOXO1 and/or ETV6; moderating splicing factor expression; or reducing or reversing cell senescence and/or re-entry to cell cycle. Ideally such a composition could be used as a therapeutic targeting an age-related disease or condition or cancer.

### SUMMARY OF THE INVENTION

In accordance with an aspect of the present invention, there is provided a composition comprising one or more intermediate non-coding RNA regulators which modulate the expression of ETV6 for use in the prevention, management, amelioration or treatment of an age-related disease or condition or cancer.

The age-related disease or condition or cancer may involve dysregulation of splicing factor expression and/or dysregulation of cellular senescence.

In accordance with a further aspect of the present invention, there is provided a composition comprising one or more intermediate non-coding RNA regulators which modulate the expression of FOXO1 for use in the prevention, management, amelioration or treatment of an age-related disease or condition or cancer, wherein the age-related disease or condition or cancer involves dysregulation of splicing factor expression and/or dysregulation of cellular senescence.

In accordance with a yet further aspect of the present invention, there is provided a composition for modulating the expression of FOXO1 and/or ETV6, the composition comprising one or more intermediate non-coding RNA regulators.

In accordance with a another aspect of the present invention, there is provided a composition for attenuating splicing factor expression, the composition comprising an expression modulator of FOXO1 and/or ETV6 or their downstream targets related to splicing factor regulation.

In accordance with yet a further aspect of the present invention, there is provided a composition for reducing or reversing cell senescence and/or re-entry to cell cycle, the composition comprising an expression modulator of FOXO1 and/or ETV6 or their downstream targets related to splicing factor regulation.

It will be apparent to the skilled addressee that the compositions of the above aspects may comprise a separate modulator of FOXO1 and a separate modulator of ETV6, or their individual or combined target genes. Alternatively, the composition may simply comprise a combined modulator of FOXO1 and ETV6 or their target genes.

The present inventors have advantageously found that modulating the expression of FOXO1 and/or ETV6 targets the activity of downstream effectors of splicing and senescence and may therefore represent promising targets for a range of future therapeutics.

The modulation of FOXO1 and/or ETV6 or their target genes in the above aspects may be by using a number of types of molecules, such as inhibitors. An inhibitor is any molecule or molecules which limits, prevents or blocks the action or function of FOXO1 and/or ETV6 or any downstream effector molecules.

The expression modulator of FOXO1 and/or ETV6 may comprise one or more intermediate non-coding RNA regulators. The one or more intermediate non-coding RNA regulators may comprise two or more intermediate non-coding RNA regulators.

The intermediate non-coding RNA regulators may comprise miRNAs, miRNA mimics or antagomiRs. In certain embodiments, the intermediate non-coding RNA regulators are selected from one or more of the following: MIR142; MIR3124; MIR3188; MIR3196; MIR320E; MIR330; MIR3675; MIR4316; MIR4488; MIR4496; MIR4513; MIR4674; MIR4707; MIR4772; MIR6088; MIR6129; MIR6780A; MIR6797; MIR6803; MIR6810; MIR6842; or MIR7155. More preferably, the intermediate non-coding RNA regulators are selected from one or more of the following: MIR3124; MIR3675; MIR4496; MIR6780A; MIR6810; MIR6842; or MIR7155.

The biochemical and functional pathways enriched for FOXO1 or ETV6 target genes are exemplified in Table A below. Potentially, disrupting any one of the genes or pathways outlined in Table A may result in the modulation of FOXO1 and/or ETV6 expression.

TABLE A
Biochemical and functional pathways enriched for FOXO1 or ETV6 target genes

| Term | P-value | Genes |
|---|---|---|
| ETV6 | | |
| Senescence-Associated Secretory Phenotype (SASP)_R-HSA-2559582 | 0.0002 | IL1A;CDKN1A;HIST1H3A;ANAPC16;H2AFX; |
| | | UBC;HIST1H2AD;HIST1H2AB |
| Mitotic Prophase_R-HSA-68875 | 0.0004 | RAB1A;HIST1H3A;PRKCB;NEK6;H2AFX; |
| | | HIST1H2AD;LPIN1;RAE1;HIST1H2AB |
| Cellular Senescence_R-HSA-2559583 | 0.0021 | IL1A;CDKN1A;HIST1H3A;ANAPC16;SP1;UBC; |
| | | H2AFX;HIST1H2AD;HIST1H1A;HIST1H2AB |
| M Phase_R-HSA-68886 | 0.0045 | RAB1A;ANAPC16;PRKCB;NEK6;H2AFX;HIST1H3A;UBC; |
| | | HIST1H2AD;KIF2C;CENPP;LPIN1;RAE1; HIST1H2AB |
| RMTs methylate histone arginines_R-HSA-3214858 | 0.0024 | SMARCC2;HIST1H3A;H2AFX;HIST1H2AD;HIST1H2AB |
| Activation of HOX genes during differentiation_R-HSA-5619507 | 0.0027 | HIST1H3A;NCOA3;H2AFX;POLR2E; |
| | | HIST1H2AD;AJUBA;HIST1H2AB |
| Activation of anterior HOX genes in early embryogenesis_R-HSA-5617472 | 0.0027 | HIST1H3A;NCOA3;H2AFX;POLR2E; |
| | | HIST1H2AD;AJUBA;HIST1H2AB |
| RNA Polymerase 1 Promoter Opening_R-HSA-73728 | 0.0034 | HIST1H3A;H2AFX;HIST1H2AD;HIST1H2AB |
| Transcriptional regulation by small RNAs_R-HSA-5578749 | 0.004 | HIST1H3A;H2AFX;POLR2E; |
| | | HIST1H2AD;RAE1;HIST1H2AB |
| Cell Cycle, Mitotic_R-HSA-69278 | 0.0091 | TOP2A;RAB1A;CDKN1A;ANAPC16;PRKCB; |
| | | NEK6;H2AFX;HAUS4;HIST1H3A; |
| | | UBC;CEP72;HIST1H2AD; KIF2C;CENPP; |
| | | LPIN1;AJUBA;RAE1;HIST1H2AB |
| FOX01 | | |
| Mitotic Prophase_R-HSA-68875 | 0.0003 | RAB1A;HIST1H3A;NEK6;HIST1H2AD; |
| | | LPIN1;RAE1;HIST1H2AB |
| Senescence-Associated Secretory Phenotype (SASP)_R-HSA-2559582 | 0.0003 | IL1A;HIST1H3A;ANAPC16;UBC;HIST1H2AD;HIST1H2AB |
| Cellular Senescence_R-HSA-2559583 | 0.0008 | IL1A;HIST1H3A;ANAPC16;SP1;UBC; |
| | | HIST1H2AD;HIST1H1A;HIST1H2AB |
| M Phase_R-HSA-68886 | 0.0017 | RAB1A;HIST1H3A;ANAPC16;NEK6;UBC; |
| | | HIST1H2AD;KIF2C;LPIN1;RAE1;HIST1H2AB |
| Cell Cycle, Mitotic_R-HSA-69278 | 0.0044 | TOP2A;RAB1A;ANAPC16;NEK6;HAUS4;HIST1H3A;UBC; |
| | | HIST1H2AD;KIF2C;LPIN1;AJUBA;RAE1;HIST1 H2AB |
| Transcriptional regulation by small RNAs_R-HSA-5578749 | 0.0018 | HIST1H3A;POLR2E;HIST1H2AD;RAE1;HIST1H2AB |
| RMTs methylate histone arginines_R-HSA-3214858 | 0.0021 | SMARCC2;HIST1H3A;HIST1H2AD;HIST1H2AB |
| Transcription-Coupled Nucleotide Excision Repair (TC-NER)_R-HSA-678182 | 0.0023 | CUL4A;COPS6;ERCC1;UBC;POLR2E |
| Cellular responses to stress_R-HSA-2262752 | 0.0054 | IL1A;HIST1H3A;ANAPC16;SP1;UBC;HSPA6; |
| | | HIST1H2AD;LAMTOR4;RAE1;HIST1H1A;HIST1H2AB |
| Formation of TC-NER Pre-Incision Complex_R-HSA-6781823 | 0.0039 | CUL4A;COPS6;UBC;POLR2E |

Cellular plasticity is a key facet of cellular homeostasis requiring correct temporal and spatial patterns of alternative splicing. Splicing factors, which orchestrate this process, demonstrate age-related dysregulation of expression, and are emerging as potential influences on ageing and longevity. The upstream drivers of these alterations are still unclear, but may involve aberrant cellular signalling.

The inventors compared the phosphorylation status of proteins in multiple signalling pathways in early and late passage human primary fibroblasts and determined the responses of ‘young’ cells to cytokines, known activators of ERK and AKT signalling. They then assessed the impact of chemical inhibition, or targeted knockdown of direct downstream targets of the ERK and AKT pathways, on splicing factor expression, cellular senescence and proliferation kinetics in senescent primary human fibroblasts.

Surprisingly, and unexpectedly, components of both ERK and AKT signalling pathways demonstrated increased activation during cellular ageing. Early passage cells exposed to cytokines also demonstrated alterations in splicing factor expression. Inhibition of AKT and ERK pathways led to upregulation of splicing factor expression, reduction in senescent cell load and reversal of multiple cellular senescence phenotypes in a dose-dependent manner.

The dose of the inhibitor of ERK and/or inhibitor of AKT in the above compositions will preferably be low. During experimentation, the inventors have unexpectedly found low dose chemical inhibition of either ERK or AKT signalling at 1 μM for 24 hours resulted in restoration of splicing factor expression to levels consistent with those seen in younger passage cells, reversal of senescence and re-entry to cell cycle for a proportion of the cells tested.

In a further aspect of the present invention, there is provided a composition capable of modulating splicing factor expression, the composition comprising one or more compounds able to bind to, with, or inhibit, FOXO1 and/or ETV6 genes. Preferably, the composition comprises one or more compounds able to bind to, or inhibit, FOXO1 and ETV6 genes (or other FOXO or ETS family member genes) or gene products thereof.

The inventors advantageously noted that the targeted knockdown of the genes encoding downstream targets FOXO1 or ETV6 was sufficient to mimic the observations found in respect of ERK and AKT inhibition.

The compositions of the above aspects may have a number of uses, from laboratory reagents and research tools to medicaments. In respect of laboratory reagents, the compositions may be used as research tools for investigating the effect of reduced gene expression of FOXO1 and/or ETV6; or restoring and/or increasing splicing factor expression; or reducing or reversing cell senescence and/or re-entry to cell cycle. The compositions may also be used as a way of reducing or reversing cell senescence and/or re-entry to cell cycle for cell culture, including stem cell culture for research and therapeutic application. The compositions could be used to increase viable number of passages in cell culture and/or reduce senescent cell populations.

The compositions may comprise inhibitors of ERK or AKT signalling.

The compositions may be in the form of a pharmaceutical preparation.

The compositions may be for use as a medicament.

Advantageously, the results produced by the inventors suggest that age-associated dysregulation of splicing factor expression and cellular senescence may derive in part from altered activity of ERK and AKT signalling, and act through the ETV6 and FOXO1 transcription factors. Targeting the activity of downstream effectors of ERK and AKT may therefore represent promising targets for future therapeutic intervention.

The compositions may be for use in the prevention, management, amelioration or treatment of an age-related disease or condition.

The compositions may be for use in a method of prevention, management, amelioration or treatment of an age-related disease or condition, the method comprising administering an therapeutically effective amount of the composition to a subject in need thereof.

In a related aspect, the invention may comprise the composition, for use in the manufacture of a medicament for the prevention, management, amelioration or treatment of an age-related disease or condition.

The age-related disease or condition may encompass a number of age-related conditions such as Alzheimer's disease, cardiovascular disease, hypertension, arthritis, osteoporosis, type 2 diabetes, cancer, Parkinson's disease, cognitive dysfunction or frailty. The age-related disease or condition may also encompass a number of conditions suffered by younger subjects who are suffering from certain conditions results in premature aging, termed progeroid syndromes—such as Werner syndrome and Hutchinson-Gilford progeria.

The compositions may be for use in the prevention, management, amelioration or treatment of cancer.

The compositions may be for use in a method of prevention, management, amelioration or treatment of cancer, the method comprising administering an therapeutically effective amount of the composition to a subject in need thereof.

In a related aspect, the invention may comprise the composition, for use in the manufacture of a medicament for the prevention, management, amelioration or treatment of cancer.

The compositions may also be for use as a nutraceutical or cosmetic product so as to reduce the effects of aging.

In accordance with a further aspect of the present invention, there is provided a related combination of an inhibitor of ERK and an inhibitor of AKT for:

a) modulating gene expression of FOXO1 and/or ETV6 or their target genes;

b) restoring and/or increasing splicing factor expression; or

c) reducing or reversing cell senescence and/or re-entry to cell cycle.

As used herein, the terms “treatment”, “treating”, “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting or slowing its development; and (c) relieving the disease, i.e., causing regression of the disease.

The term “subject” used herein includes any human or nonhuman animal. The term “nonhuman animal” includes all mammals, such as nonhuman primates, sheep, dogs, cats, cows, horses.

A “therapeutically effective amount” refers to the amount of composition that, when administered to a subject for treating a disease, is sufficient to affect such treatment for the disease. The “therapeutically effective amount” will vary depending on the pharmaceutically active ingredient used, the disease and its severity and the age, weight, etc., of the subject to be treated.

In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrathecal, and intravenous routes, i.e., any route of administration other than through the alimentary canal. Parenteral administration can be carried to effect systemic or local delivery. Where systemic delivery is desired, administration typically involves invasive or systemically absorbed topical or mucosal administration of pharmaceutical preparations. Enteral routes of administration include, but are not necessarily limited to, oral and rectal (e.g., using a suppository) delivery.

Conventional and pharmaceutically acceptable routes of administration include intranasal, intramuscular, intra-tracheal, intrathecal, intracranial, subcutaneous, intradermal, topical, intravenous, intraperitoneal, intra-arterial (for example, via the carotid artery), spinal or brain delivery, rectal, nasal, oral, and other enteral and parenteral routes of administration.

In some embodiments, a composition of the invention, or a combination of the invention, may be administered with one or more other compounds effective for the prevention, management, amelioration or treatment of an age-related disease or condition or cancer.

In an alternative aspect of the present invention, there is provided the use of a composition comprising one or more intermediate non-coding RNA regulators which modulate the expression of ETV6 for the cosmetic treatment of the effects of ageing. The effects of aging may involve dysregulation of splicing factor expression and/or dysregulation of cellular senescence.

In a yet further aspect of the present invention, there is provided the use of a composition comprising one or more intermediate non-coding RNA regulators which modulate the expression of FOXO1 for the cosmetic treatment of the effects of ageing, where the effects of aging involves dysregulation of splicing factor expression and/or dysregulation of cellular senescence.

The term “cosmetic treatment” is intended to mean any non-medical treatment which may be systemically or topically applied to a human or animal.

The term, “effects of aging” is intended to mean progressive (but not disease-based) physiological changes in a human or animal that lead to senescence, or a decline of biological functions and its ability to adapt to metabolic stress.

The modulator or modulators of FOXO1 and/or ETV6 or their target genes may be artificially generated. That is to say that it is not naturally occurring. The modulator or modulators of FOXO1 and/or ETV6 and their target genes may however be a naturally occurring molecule or molecules whose concentration and formulation in a medicament or pharmaceutical preparation or combination enables it to be used for the prevention, management, amelioration or treatment of an age-related disease or condition or cancer, whereas otherwise it would have no or limited efficacy. Whilst the inhibitor or inhibitors may be a naturally occurring molecule or molecules, it will be understood that the concentration and formulation of the molecule or molecules found to be therapeutically effective would not be present in nature at such a concentration or in a formulation with other components.

The inhibitor or inhibitors may comprise an antibody or antibodies or antibody mixture. Such antibody or antibodies may be polyclonal or may be monoclonal. It will be apparent to the skilled addressee how to produce antibodies which would act as inhibitors. Preferably the antibodies will be humanised.

In other embodiments, the inhibitor or inhibitors comprise a peptide or peptide mimetic thereof, or C-terminal amidated peptide thereof.

The terms “peptide” and “peptides” include compounds that have amino acid residues (H—Cα-[side chain]) but which may be joined by peptide (—CO—NH—) or non-peptide linkages.

Peptides may be synthesised by the Fmoc-polyamide mode of solid-phase peptide synthesis.

The peptide may be a peptide aptamer. Peptide aptamers typically consist of short, 5-20 amino acid residues long sequences that can bind to a specific target molecule.

There are a number of different approaches to the design and synthesis of peptide composition that do not contain amide bonds. In one approach, one or more amide bonds are replaced in an essentially isoteric manner by a variety of chemical functional groups.

Retro-inverso peptidomimetics, in which the peptide bonds are reversed, can be synthesised by methods known in the art. This approach involves making pseudopeptides containing changes involving the backbone, and not the orientation of side chains. Retro-inverse peptides, which contain NH—CO bonds instead of CO—NH peptide bonds, are more resistant to proteolysis.

The peptide may be linear. Although, it may be advantageous to introduce a cyclic moiety into a peptide-based framework. The cyclic moiety restricts the conformational space of the peptide structure and this may lead to an increased efficacy. An added advantage of this strategy is that the introduction of a cyclic moiety into a peptide may also result in the peptide having a diminished sensitivity to cellular peptidases.

In some embodiments of the invention the peptide may be joined to another moiety. Convenient moieties to which the peptide may be joined include polyethylene glycol (PEG) and peptide sequences, such as TAT and antennapedia which enhance delivery to cells.

In some embodiments, the inhibitor or inhibitors is/are pro-drugs of the peptide. A pro-drug is a compound which is metabolised in vivo to produce the molecule, such as a protein. One of skill in the art will be familiar with the preparation of pro-drugs.

The peptide may be a peptide mimetic. A peptide mimetic is an organic compound having similar geometry and polarity to the molecules defined herein, and which has a substantially similar function. A mimetic may be a molecule in which the NH groups of one or more peptide links are replaced by CH₂ groups. A mimetic may be a molecule in which one or more amino acid residues is replaced by an aryl group, such as a napthyl group.

In other embodiments, an inhibitor or inhibitors comprise nucleic acid, such as single stranded DNA or RNA, which is capable of binding to and inhibiting downstream effectors of FOXO1 and/or ETV6 or their target genes. It is envisaged that the same targets are also suitable for targeting with peptides and peptide aptamers will also be suitable for targeting with RNA or modified RNA aptamers. Nucleic acids such as single stranded DNAs and RNAs may be provided that bind to and inhibit downstream effectors of FOXO1 and/or ETV6 or their target genes. Typically, the nucleic acids are single stranded and have from 100 to 5000 bases.

In yet other embodiments, an inhibitor or inhibitors comprise a small molecule or small molecules.

It will be apparent to the skilled addressee that if the composition is intended to comprise one or more compounds able to bind to, or modulate FOXO1 and/or ETV6 gene expression or gene products thereof.

Features, integers, characteristics, compounds, molecules, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and figures), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### DETAILED DESCRIPTION OF THE INVENTION

Embodiments of the invention are described below, by way of example only, with reference to the accompanying figure in which:

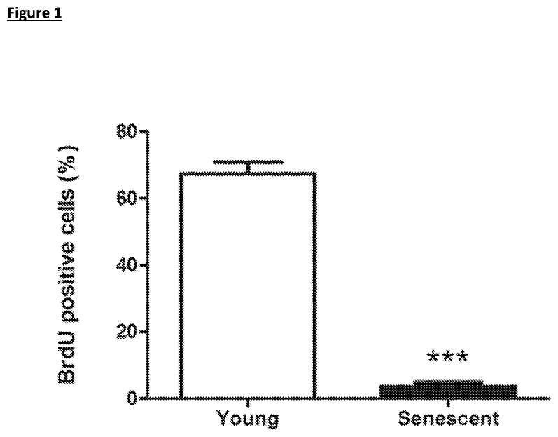

FIG. 1 shows the differences in proliferative cell fraction between early passage and late passage (senescent) cell cultures. Early and late passage cell populations were subjected to a 24 hr label with the S-phase marker BrdU, to selectively stain actively growing cells. ***=p=<0.0001, n=3 biological replicates, 300 nuclei counted per replicate;

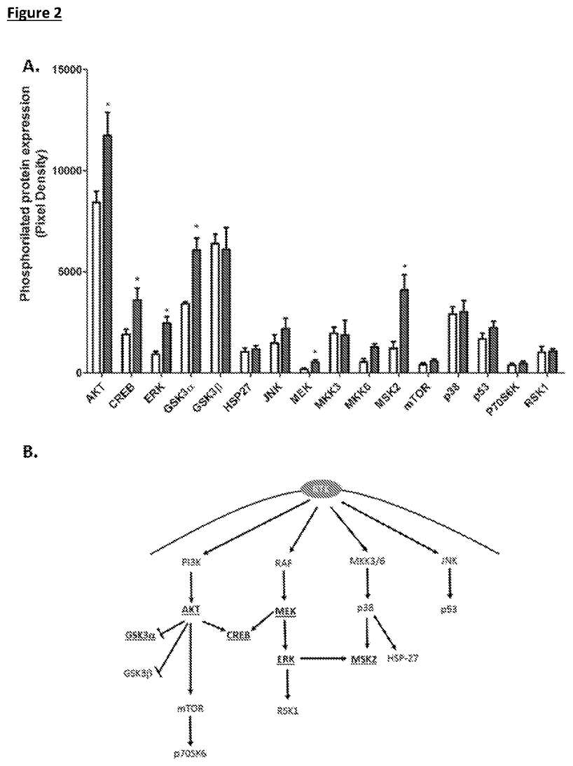

FIG. 2 shows age-related changes in protein phosphorylation for targets in the ERK and AKT pathways. A. Is a graph showing phosphorylated protein expression of key targets from AKT and ERK pathways were assessed in young (PD=25) and senescent (PD=63) cells. White and grey bars represent young and senescent cell lysates respectively. B. Is a schematic diagram showing the genes tested for senescence-related phosphorylation differences in the AKT and ERK pathways. Targets showing significantly different levels of phosphorylation are highlighted in bold underlined text. Targets showing no significant differences in protein phosphorylation are indicated in normal typeface. Statistical significance is indicated by stars with *p<0.05. Error bars represent the standard error of the mean;

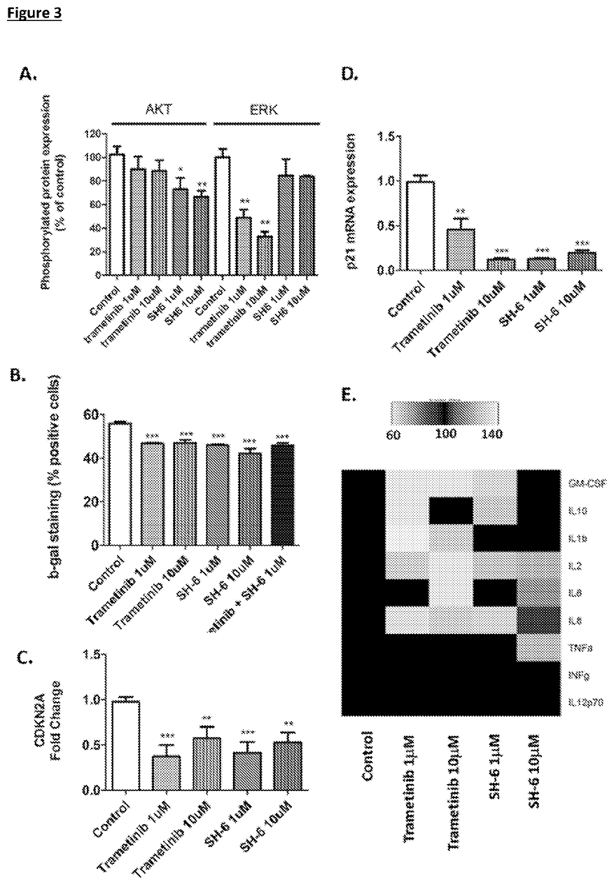

FIG. 3 shows chemical inhibition of ERK or AKT signalling is associated with rescue from cellular senescence phenotypes. A. The proportion of cells staining positive for Senescence associated β-galactosidase (SA-β-Gal) activity following treatment with trametinib and SH-6 at 1 or 10 μM was determined by manually counting the percentage of SA-β gal positive cells. N=>300 cells for each sample. B. Levels of senescence-associated transcript CDKN2A, which encodes the senescence marker p16 was assessed in senescent cells by qRTPCR. Data are expressed relative to stable endogenous control genes GUSB, IDH3B and PP/A, and are given normalised to the levels of the individual transcripts as present in vehicle-only treated control cells. Fold change was calculated for in triplicate for three biological replicates. Statistical significance is indicated by ** p<0.005, *** p<0.0001 (2 way ANOVA). C. Protein levels of various pro-inflammatory SASP factors following treatment with ERK and AKT inhibitors at 1 μM and 10 μM. D. Expression levels of CDKN1A transcripts encoding the DNA damage response protein p21. E. Is a heat map indicating fold changes in SASP protein expression. Green indicates up-regulation whilst red denotes down-regulation. Only statistically significant changes are presented in the heat map. The colour scale refers to percentage change in expression. Experiments were carried out in duplicate for a total of 6 biological replicates. Error bars represent the standard error of the mean;

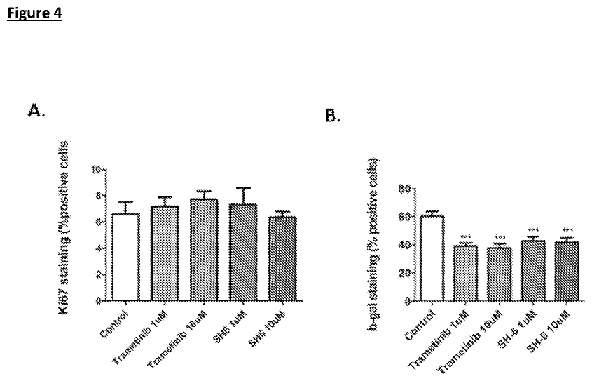

FIG. 4 shows the effect of ERK or AKT inhibitors on splicing factor expression and senescent cell load under conditions that do not permit cell proliferation. To establish whether apparent ‘rejuvenation’ of senescent cell cultures was derived from altered proliferation kinetics of non-senescent cells in the culture, or arise from a genuine rescue in response to treatment, selected experiments were repeated under conditions of serum starvation, where cells were prevented from dividing. A. Ki67 staining showed cells were non-proliferative, whereas B. SA-b-Gal staining revealed rescue was still evident. ***=p=<0.0001, n=3 biological replicates, 300 nuclei counted per replicate;

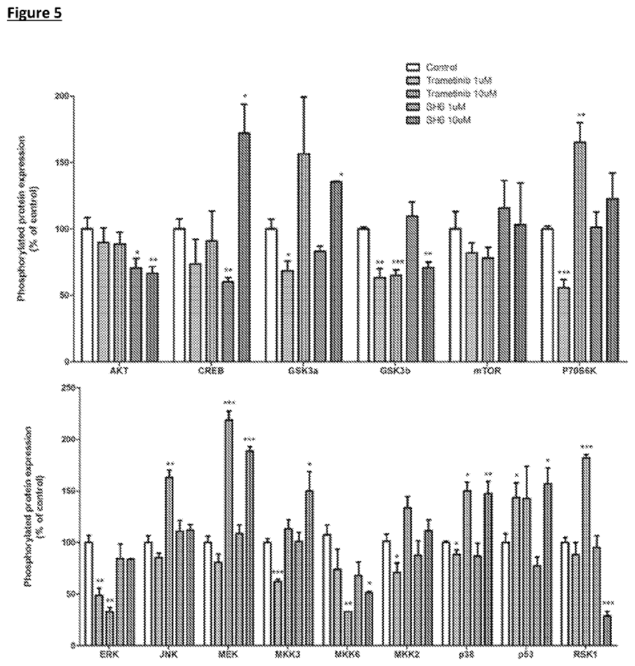

FIG. 5 shows changes in the phosphorylation status of ERK and AKT signalling proteins, and also proteins in linked signalling pathways in response to low dose ERK or AKT inhibition. White, light grey, light grey hatched, dark grey and dark grey hatched boxes represent controls, low dose trametinib, high dose trametinib, low dose SH-6 and high dose SH-6 respectively. Statistical significance is indicated by stars with *p<0.05. Error bars represent the standard error of the mean. ***=p=<0.0001. Data are derived from 3 biological and 2 technical replicates;

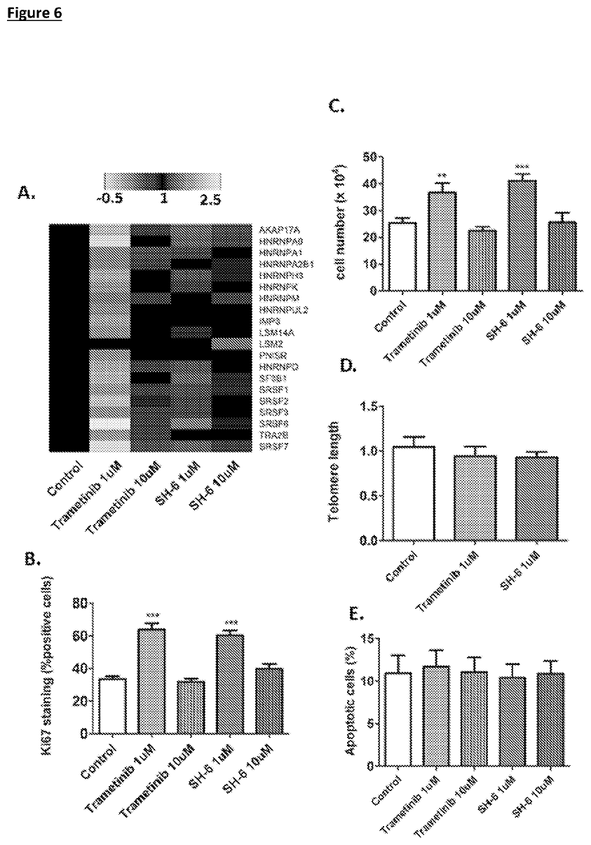

FIG. 6 shows inhibition of AKT or ERK pathways affects splicing factor transcript expression and cell proliferation rate. A. The change in splicing factor mRNA levels in response to 24 hr treatment with ERK or AKT inhibitors at 1 μM and 10 μM are given. Green indicates up-regulated genes, red denotes down-regulated genes. The colour scale refers to fold-change in expression. Only statistically significant changes are presented in the heat map B. Proliferation index was assessed for treated cells as assessed by Ki67 immunofluorescence (>400 nuclei counted per sample). C. Cell counts following treatment with 1 μM and 10 μM ERK or AKT inhibitors. D. Telomere length as quantified by qPCR relative to the 36B4 endogenous control and normalised to telomere length in vehicle-only control. E. Apoptotic index in senescent cells treated with inhibitors as determined by TUNEL assay. Data are derived from duplicate testing of 3 biological replicates. Statistical significance is indicated by ** p<0.005, *** p<0.0001. Error bars represent the standard error of the mean;

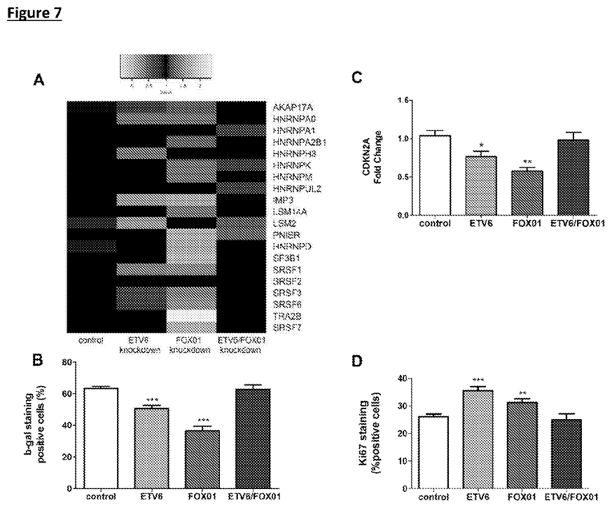

FIG. 7 shows the cellular and molecular effects of targeted knockdown of ETV6 and FOXO1 genes. A. Levels of splicing factor expression following FOXO1, ETV6 or ETV6/FOXO1 gene knockdown. Green indicates up-regulated genes, red denotes down-regulated genes. The colour scale refers to fold-change in expression. Only statistically significant changes are presented in the heat map B. Senescent cell load as indicated by SA-β-Gal following FOXO1, ETV6 and ETV6/FOXO1 gene knockdown. n>300 cells for each sample. C. Senescent cell load as indicated by CDKN2A gene expression following FOXO1, ETV6 and ETV6/FOXO1 gene knockdown. Data are expressed relative to stable endogenous control genes GUSB, IDH3B and PP/A, and normalised to the levels of the individual transcripts in vehicle only controls. D. Proliferation index was assessed following FOXO1, ETV6 and ETV6/FOXO1 gene knockdown by Ki67 immunofluorescence (>400 nuclei counted per sample). E. The effect of FOXO1 or ETV6 gene knockdown on reciprocal expression of ETV6 and FOXO1 genes respectively. Data are expressed relative to stable endogenous control genes GUSB, IDH3B and PP/A, and normalised to the levels of the individual transcripts in control. Data are derived from duplicate testing of 3 biological replicates. Statistical significance is indicated by *p<0.05, ** p<0.005, *** p<0.0001. Error bars represent the standard error of the mean;

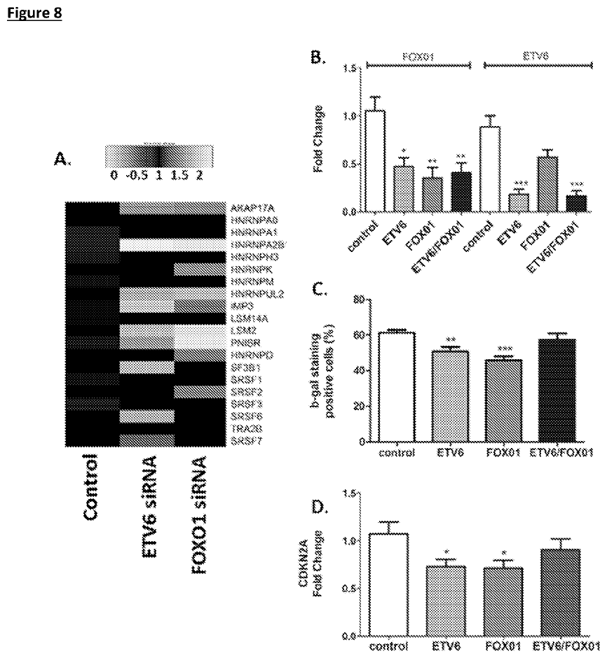

FIG. 8 shows the effect of FOXO1 and/or ETV6 knockdown achieved by a second methodology, siRNA. The effects of ETV6 or FOXO1 gene knockdown were confirmed by siRNA against the genes in question. Levels of knockdown were determined to be 43% for ETV6 and 65% for FOXO1. The effects of gene knockdown on splicing factor expression (A), ETV6 and FOXO1 expression (B); the identity of the gene manipulated is given on the X axis. The first 4 bars refer to effects on FOXO1 gene expression and the latter to effects on ETV6 expression. Effects on senescent cell load (C) and CDKN2A expression (D) were also examined. Only statistically significant changes are presented in the heat map ***=p=<0.0001, n=3 biological replicates. Data are from 3 independent biological replicates;

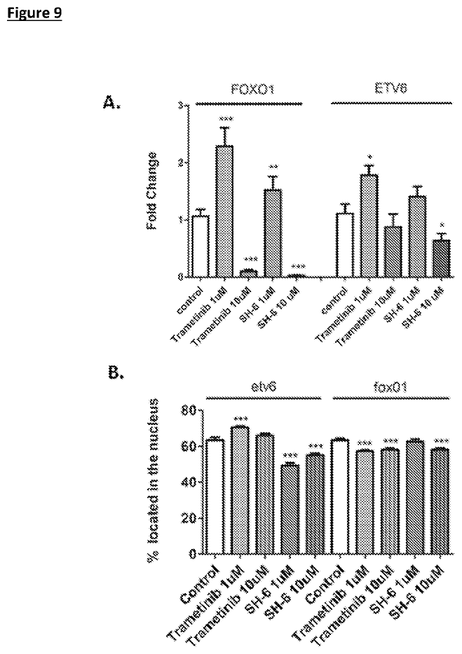

FIG. 9 shows the effect of ERK or AKT inhibition on FOXO1 and ETV6 transcript expression and subcellular localisation. A. The effects of ERK and AKT inhibition on ETV6 and FOXO1 expression; The treatment is indicated on the X axis. The first 5 bars refer to effects on FOXO1 expression, the second 5 bars refer to effects on ETV6 expression. Data are from 3 independent biological replicates each with 3 technical replicates. B. The effects of ERK and AKT inhibition on ETV6 and FOXO1 protein subcellular localisation; the treatment is indicated on the X axis. The first 5 bars refer to effects on ETV6 localisation, the second 5 bars refer to effects on FOXO1 localisation. ***=p=<0.0001. Data are from at least 50 cells from each of 3 independent biological replicates; and

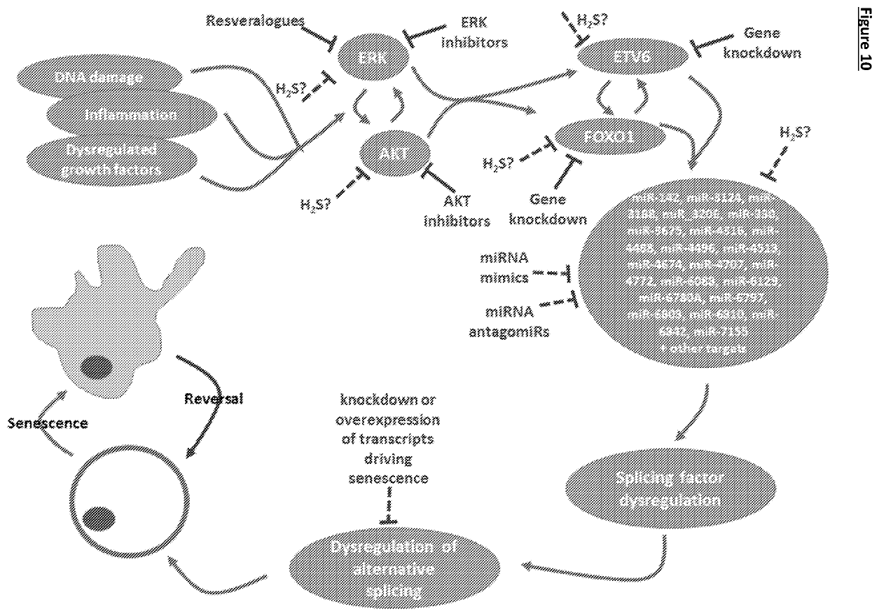

FIG. 10 is a schematic diagram showing the indirect control of senescence as elucidated by the experiments conducted by the inventors.

### EXAMPLE

The aims of the experiments in the example were to study the effect of manipulation of ERK and AKT signalling pathways by chemical inhibition or targeted gene knockdown on splicing factor expression and cellular senescence and proliferation kinetics in late passage human primary fibroblasts.

### 1. Increased Phosphorylation of Target Proteins in the MEK/ERK and PI3K/AKT Pathways in Senescent Cells

Cell cultures were considered senescent when the population doubling time had slowed to <0.5 cell divisions per week. At this point, ^{˜}3% of cells stained positive for the 5-phase marker BrdU, which indicates actively replicating DNA (FIG. 1). Late passage primary human dermal fibroblasts were found to have reached senescence at PD=63. Earlier passage cells were at PD=25. We noted increased phosphorylation was noted for AKT, CREB, ERK, GSK3α, MEK, and MSK2 signalling proteins in late passage fibroblasts, whereas no increase in phosphorylation of GSK3β, HPS27, JNK, MKK3, MKK6, mTOR, p38, p58, p70SK6 or RSK1 proteins was apparent in these cells (FIG. 2A). Several of these targets lie in the AKT or ERK signalling pathways (FIG. 2B).

### 2. Inhibition of MEK/ERK and PI3K/AKT Pathways Rescue Cellular Senescence Phenotypes

We assessed the effects of targeted inhibition of AKT or MEK/ERK in late passage human primary fibroblasts using specific inhibitors. Treatment with SH-6 (AKT) or Trametinib (MEK/ERK) at both 1 μM and 10 μM resulted in a specific reduction in AKT or ERK protein phosphorylation for SH-6 and trametinib respectively, at both high and low dose (FIG. 3A). This was accompanied by a small but robust decrease in the senescent cell population as indicated by reduced SA-β-Gal staining (p=<0.0001; FIG. 3B) and a decrease in the expression of both p16 and p21 (FIG. 3C and FIG. 3D). Reduction in levels of several secreted SASP proteins were also noted in cells treated with both low and high dose trametinib and SH-6 (table 1 below; FIG. 3E).

TABLE 1
The changes in SASP protein expression in response to MEK/ERK or AKT inhibition

| | Control | Trametinib 1 uM | | Trametinib 10 uM | | SH-6 1 uM | | SH-6 10 uM | |
|---|---|---|---|---|---|---|---|---|---|
| GM-CSF | 100 | 24.65*** | (1.76) | 9.85*** | (0.99) | 47.63** | (2.27) | 129.67 | (10.16) |
| IL10 | 100 | 34.37*** | (11.05) | 86.01 | (29.30) | 52.94** | (5.07) | 97.78 | (25.08) |
| IL1b | 100 | 156.70** | (26.51) | 50.35** | (4.12) | 115.68 | (21.8) | 84.22 | (9.52) |
| IL2 | 100 | 50.72** | (17.36) | 14.39*** | (2.56) | 52.78* | (11.25) | 58.26* | (12.62) |
| IL6 | 100 | 77.75 | (2.63) | 33.57*** | (3.57) | 83.47 | (0.93) | 66.18* | (11.87) |
| IL8 | 100 | 44.42** | (2.43) | 5.33*** | (0.51) | 48.21** | (0.88) | 88.68 | (5.41) |
| TNFa | 100 | 104.61 | (8.05) | 118.65 | (6.92) | 109.81 | (6.83) | 164.04** | (15.86) |
| INF-g | 100 | 95.08 | (9.81) | 106.33 | (6.39) | 96.65 | (1.11) | 117.02 | (6.79) |
| IL12p70 | 100 | 69.64 | (4.40) | 77.78 | (3.02) | 67.45 | (3.14) | 100.47 | (10.97) |

(Protein levels of pro-inflammatory components of the senescence associated secretory phenotype (SASP) were determined using Mesoscale ELISA platform in the culture medium from senescent cells treated with the ERK inhibitor trametinib or the AKT inhibitor SH-6 at 1 μM or 10 μM for 24 hrs. The results are expressed as a percentage of the control value (vehicle only) of 3 independent experiments. Standard error of the mean (SEM) is given in parentheses. Statistical significance is indicated by stars with
*p < 0.05,
**p < 0.005 and
***p < 0.001 compared with the corresponding control value.)

To establish whether the drop in the senescent cell fraction was due to active ‘rescue’, or merely due to increased proliferation of the growth arrested cells in the culture, we repeated the treatments under conditions of serum starvation, which inhibits cell proliferation. An approximately 20% reduction of SA-β-Gal positive cells was also noted in serum starved cells, indicating that the effect was not derived merely from altered culture division kinetics (FIG. 4). This is in accordance with our previous findings (Latorre et al. 2017).

### 3. Inhibition of MEK/ERK and PI3K/AKT Alters Target Protein Phosphorylation Status Both Upstream and Down-Stream of ERK and AKT.

To investigate potential downstream targets which might be influencing cellular senescence, we assessed the effect of ERK and AKT inhibition on downstream signalling targets. As expected, changes to the phosphorylation levels of ERK and AKT in response to both high and low dose pathway inhibition were observed, along with changes to the phosphorylation status of GSK3α, GSK3β, p70SK6 and CREB downstream targets. Down regulation of GSK3β was evident for both high and low concentrations of trametinib, but high dose SH-6 only, whereas GSK3a phosphorylation demonstrated decreased levels at low dose and increased levels at high dose for both trametinib and SH-6 alone, although results were only statistically significant for high dose SH6 and low dose trametinib (FIG. 5A). This pattern of antagonistic action was also apparent for upstream kinases including p38. Several other upstream targets including p53, MKK3, MKK6, and MEK demonstrated differences in phosphorylation status after treatment, with effects being most commonly at high dose (FIG. 5B). Altered p53 phosphorylation is consistent with cells being restored to a more proliferative and less senescent state by treatment with AKT or ERK inhibitors (Pise-Masison et al. 1998).

### 4. ERK and AKT Pathways Influence Splicing Factor Expression

Dysregulated splicing factor expression have been implicated in cellular senescence and ageing in human populations and cells (Harries et al. 2011; Holly et al. 2013) and in animal models (Heintz et al. 2016; Lee et al. 2016). Furthermore, restoration of splicing factor expression using small molecules has been associated with rescue of cellular senescence in our previous work (Latorre et al. 2017). We therefore assessed the expression of an a priori list of 20 splicing regulator genes previously demonstrated to show senescence-related changes in late passage human fibroblasts treated with low (1 μM) and high (10 μM) dose AKT and ERK inhibitors (SH-6 and trametinib respectively). Treatment with both trametinib and SH-6 at low dose (1 μM) was associated with upregulation of multiple splicing factors (FIG. 6A; table 2 below).

TABLE 2
Changes in splicing factor expression in response to low (1 μM)
and high (10 μM) dose MEK/ERK or AKT inhibitors.

| | Control | Trametinib 1 uM | | Trametinib 10 uM | | SH-6 1 uM | | SH-6 10 uM | |
|---|---|---|---|---|---|---|---|---|---|
| AKAP17A | 1.00 (0.02) | 2.01*** | (0.21) | 0.74** | (0.04) | 1.38*** | (0.07) | 0.51*** | (0.05) |
| HNRNPA0 | 1.00 (0.01) | 2.38*** | (0.16) | 1.11 | (0.15) | 1.27*** | (0.05) | 0.64*** | (0.03) |
| HNRNPA1 | 1.00 (0.01) | 1.52* | (0.34) | 0.49*** | (0.09) | 1.17*** | (0.04) | 1.01 | (0.09) |
| HNRNPA2B1 | 1.00 (0.01) | 1.53* | (0.25) | 0.70*** | (0.02) | 0.98 | (0.06) | 0.88* | (0.07) |
| HNRNPH3 | 1.00 (0.01) | 1.89*** | (0.17) | 0.95 | (0.03) | 1.20** | (0.07) | 0.88* | (0.05) |
| HNRNPK | 1.00 (0.02) | 1.68*** | (0.13) | 1.02 | (0.06) | 1.14** | (0.04) | 0.99 | (0.07) |
| HNRNPM | 1.00 (0.01) | 1.46* | (0.22) | 0.69*** | (0.05) | 1.05 | (0.05) | 0.68** | (0.01) |
| HNRNPUL2 | 1.00 (0.01) | 1.63*** | (0.14) | 0.94 | (0.05) | 1.06 | (0.06) | 1.04 | (0.07) |
| IMP3 | 1.00 (0.01) | 1.77*** | (0.26) | 0.97 | (0.03) | 0.99 | (0.02) | 0.93 | (0.05) |
| LSM14A | 1.00 (0.01) | 1.61** | (0.26) | 1.18 | (0.07) | 1.10* | (0.04) | 0.89 | (0.08) |
| LSM2 | 1.00 (0.04) | 0.85 | (0.14) | 0.82 | (0.10) | 0.93 | (0.02) | 1.43* | (0.28) |
| PNISR | 1.00 (0.02) | 1.63** | (0.21) | 1.11 | (0.03) | 0.99 | (0.02) | 0.99 | (0.13) |
| HNRNPD | 1.00 (0.03) | 1.79* | (0.38) | 0.75** | (0.07) | 1.21*** | (0.15) | 0.95 | (0.13) |
| SF3B1 | 1.00 (0.02) | 1.93*** | (0.14) | 0.96 | (0.07) | 1.29*** | (0.03) | 0.86* | (0.07) |
| SRSF1 | 1.00 (0.01) | 1.69* | (0.37) | 0.68** | (0.14) | 1.19*** | (0.03) | 0.83** | (0.05) |
| SRSF2 | 1.00 (0.02) | 2.07** | (0.42) | 0.86* | (0.04) | 1.16* | (0.08) | 0.93 | (0.20) |
| SRSF3 | 1.00 (0.03) | 1.64*** | (0.10) | 0.72*** | (0.02) | 1.17*** | (0.04) | 0.92 | (0.05) |
| SRSF6 | 1.00 (0.02) | 2.68*** | (0.47) | 0.69*** | (0.03) | 1.49*** | (0.07) | 0.88* | (0.05) |
| TRA2B | 1.00 (0.02) | 1.66* | (0.33) | 0.73** | (0.11) | 1.09 | (0.07) | 0.94 | (0.13) |
| SRSF7 | 1.00 (0.01) | 2.38*** | (0.32) | 0.57*** | (0.02) | 1.26** | (0.07) | 0.71*** | (0.04) |

(Changes to mRNA levels in senescent primary human fibroblasts in response to treatment with ERK inhibitors (trametinib) or AKT inhibitors (SH-6) at 1 μM or 10 μM for 24 hrs. Data are derived from duplicate testing of 3 biological replicates. Standard error of the mean (SEM) is given in parentheses. Statistical significance is indicated by stars with
*p < 0.05,
**p < 0.005 and
***p < 0.0001 compared with the corresponding control value.)

Effects were more marked for MEK/ERK inhibited cells than for AKT inhibited cells. Surprisingly, high dose inhibitors (10 μM) demonstrated an antagonistic effect on splicing factor expression (FIG. 6A; table 2). This dose-dependent response was also noted for proliferation kinetics (FIG. 6B,C). Low dose trametinib led to a 31% increase in proliferative index, whereas low dose SH-6 resulted in a 27% increase in Ki67 staining (p=<0.0001 and <0.0001 respectively). High dose treatment with either inhibitor resulted in no reactivation of proliferation (FIG. 6B, C). These data demonstrate a clear link between re-entry to cell cycle and splicing factor expression. No rescue of telomeres was apparent after any treatment (FIG. 7D) and no increase in apoptotic index was noted with any treatment (FIG. 7E).

### 5. ETV6 and FOXO1 are Regulators of Splicing Factor Expression and Cell Senescence Phenotypes.

ERK and AKT signalling have multiple downstream effector pathways, with significant evidence of crosstalk and autoregulation (Rhim et al. 2016). In order to clarify mechanism, it was necessary to identify the downstream effector genes to give a cleaner assessment of phenotype upon manipulation. Targeted deletion of the Foxo and Aop genes have been reported to be associated with increased lifespan in D. Melanogaster (Slack et al. 2015), and the closest human homologues of these are FOXO1 and ETV6 (Jousset et al. 1997; Kramer et al. 2003). Targeted gene knockdown with morpholino oligonucleotides revealed that down-regulation of either FOXO1 or ETV6 in late passage human primary fibroblasts resulted in a similar rescue of splicing factor gene expression to that seen with AKT or ERK inhibition, although this time the results were more marked for FOXO1 (FIG. 7A). Changes in splicing factor expression were accompanied by an approximate 18% and 40% reduction in senescent cell load as measured by SA-β-Gal staining for ETV6 and FOXO1 knockdown respectively (p=<0.0001 and 0.0001; FIG. 7B) and a corresponding 24% and 43% reduction in the expression of CDKN2A expression was also noted (p=<0.05 and <0.005 for ETV6 and FOXO1 respectively; FIG. 7C). This represents a more marked change than that noted with inhibition of either ERK or AKT signalling as a whole. Again, as we noted for splicing factor expression, effects were strongest in cells where FOXO1 expression had been manipulated. Cell proliferation was also restored in late passage fibroblasts in which expression of ETV6 or FOXO1 had been abrogated. ETV6 knockdown led to a 36% increase in proliferative index, whereas abrogation of FOXO1 resulted in a 19% increase (p=<0.0001 and <0.005 respectively; FIG. 4D). The effects of ETV6 or FOXO1 gene knockdown was also confirmed at the level of splicing factor expression, cell senescence and CDKN2A expression by the use of siRNAs targeted against the genes in question (FIG. 8).

### 6. Significant Cross-Regulation Exists Between AKT and ERK Signalling Pathways

Cross-regulatory relationships were evident between both ERK and AKT signalling pathways, and also between ETV6 and FOXO1 themselves; dual knockdown of both ETV6 and FOXO1 genes caused abrogation of rescue from multiple senescence phenotypes (FIG. 7B-D). Part of the co-regulation may partly be at the level of transcriptional interaction. ETV6 gene knockdown affected both ETV6, and FOXO1 gene expression, whereas FOXO1 gene knockdown was associated with down-regulation of FOXO1 levels alone (FIG. 8B). A similar cross-regulation is noted in the response of FOXO1 and ETV6 gene expression to ERK or AKT inhibitors. Treatment of senescent cells with low dose trametinib yielded induction of both ETV6 and FOXO1 expression at the mRNA level, whilst treatment with low dose SH-6 caused upregulation of FOXO1 alone (FIG. 9A). This may represent a compensatory counter-regulation at the level of mRNA expression although this remains to be established. High doses of SH-6 caused a significant down-regulation of both FOXO1 and ETV6 expression, whereas higher doses of trametinib were associated with altered FOXO1 expression only (FIG. 9A). Trametinib or SH-6 treatment also caused some small but significant changes in the subcellular localisation of ETV6 and FOXO1 proteins. Low dose trametinib was associated with more nuclear retention of ETV6 protein, but less nuclear FOXO1 protein. High dose trametinib was associated with less nuclear FOXO1 protein alone. Low dose SH-6 was associated with significantly lower ETV6 nuclear retention but FOXO1 subcellular localisation was unaffected. High dose SH-6 caused reductions in nuclear localisation of both ETV6 and FOXO1 proteins (FIG. 9B). These data indicate that interplay between FOXO1 and ETV6 occurs at the level of both transcription and protein localisation, as well as cross regulation at the level of protein activity as presented here and in previously published data (Rhim et al. 2016). Cross regulation is typical of these signalling pathways and merits future exploration.

### 7. Splicing Factors are Indirect Targets of FOXO1 and ETV6.

The target genes of FOXO1 and ETV6 were compared by analysis of publically-available Chromatin immunoprecipitation (ChIP) datasets from human cell types. 419 genes were identified that were targets of ETV6 and 242 which were targets of FOXO1. All 242 targets of FOXO1 were also targets of ETV6. Two splicing factors (HNRNPF and HNRNPLL) were direct targets of ETV6, but most splicing factors were not directly targeted, suggesting that their regulation by these proteins is indirect. FOXO1 and ETV6 target genes comprised several molecular functions, but surprisingly, almost a quarter of genes (58/242) targeted by both ETV6 and FOXO1 comprised non-coding RNA regulators (miRNAs, snoRNAs, IncRNAs), transcription factors or cell signalling proteins (Table 3 below).

TABLE 3
Common target genes of FOXO1 and ETV6 identified
from publically-available ChIP datasets

| Gene | Function |
|---|---|
| ACRBP | Acrosome binding protein |
| ADIPOQ-AS1 | ncRNA |
| AGFG2 | Nucleocytoplasmic transport of |
| | RNAs and proteins |
| AGTRAP | Negative regulation of angiotensin |
| AJUBA | CDK phosphorylation |
| ANAPC16 | involved in mitosis |
| ANKRD52 | regulatory subunit of protein phosphatase 6 |
| AP4B1 | Targets proteins to lysosomes |
| AP4B1-AS1 | ncRNA |
| APOA1BP | Interacts with apolipoprotein A-I |
| ARHGAP25 | Cell signalling |
| ARL8B | Potential role in lysosome motility |
| ASCC1 | Transcriptional coactivator |
| ATF3 | Transcription factor |
| BHLHA15 | Transcriptional regulator |
| BIN3 | Intracellular transport |
| BRD3 | Ser/Thr kinase |
| BZRAP1-AS1 | ncRNA |
| C16orf70 | unknown function |
| C2orf42 | unknown function |
| C4BPA | Activation of complement cascade |
| CCDC153 | unknown function |
| CD27 | TNFα family submember |
| CD27-AS1 | ncRNA |
| CD4 | Interaction with MHC |
| CD44 | Cell:Cell interactions |
| CD79A | Ig-alpha component of B-cell antigen |
| CEACAM3 | Related to innate immune system |
| CENPBD1P1 | Pseudogene |
| CLCN6 | Ion channel protein |
| CLTC | component of intracellular organelles |
| COPS6 | Signalling protein |
| CST6 | Cysteine protease inhibitor |
| CTCFL | Transcription regulator |
| CUL4A | DNA damage response |
| CYB561A3 | Oxidoreductase activity |
| DAK | dihydroxyacetone kinases |
| DCLRE1B | DNA repair |
| DCTPP1 | dCTP pyrophosphatase |
| DMTF1 | Regulation of cell cycle |
| DPAGT1 | Glycoprotein biosynthesis |
| DRAP1 | Regulation of transcriptional initiation |
| EGR3 | Transcription factor |
| ELMSAN1 | Chromatin binding |
| EML2 | Cell division |
| EMR2 | G coupled protein receptor |
| EPS15 | Signalling protein |
| ERCC1 | DNA repair |
| ERVK13-1 | ncRNA |
| EZH1 | Gene regulation |
| FAM63A | Deubiquitinase |
| FIZ1 | Transcriptional regulator |
| FLJ42969 | Unknown function |
| FMNL1 | Cell division |
| G6PC | Glycolytic enzyme |
| GADD45B | Cell Cycle Regulator |
| GAL3ST4 | Galactosidase |
| GNAI2 | Signalling protein |
| GPI | Glucose-6-Phosphate isomerase |
| GPIHBP1 | Lipolytic processing |
| GPR162 | Signalling protein |
| GRTP1 | Signalling protein |
| GTF2E2 | Transcription factor |
| HAUS4 | Cell Cycle |
| HCST | Signalling protein |
| HDC | Histidine decarboxyllase |
| HIST1H1A | Histone protein |
| HIST1H2AB | Histone protein |
| HIST1H2AD | Histone protein |
| HIST1H2BF | Histone protein |
| HIST1H3A | Histone protein |
| HIST1H3D | Histone protein |
| HIST1H3E | Histone protein |
| HIST1H4B | Histone protein |
| HMBS | Hydroxymethylbilane Synthase |
| HSPA6 | Molecular chaparone |
| IKZF4 | Transcription factor |
| IL19 | cytokine |
| IL1A | cytokine |
| INPP5B | Signalling protein |
| IRF1 | Transcription factor |
| IRF2BPL | Transcription factor |
| KCNH6 | Ion channel |
| KIAA1522 | Unknown function |
| KIAA1683 | Unknown function |
| KIF2C | Cell Division |
| KISS1R | Signalling protein |
| KLK11 | Serine protease |
| LAMTOR4 | Signalling protein |
| LBHD1 | Unknown function |
| LIME1 | Signalling protein |
| LIMS1 | Signalling protein |
| LINC00114 | long non-coding RNA |
| LINC00671 | long non-coding RNA |
| LINC00881 | long non-coding RNA |
| LINC01348 | long non-coding RNA |
| LINC01573 | long non-coding RNA |
| LOC100129148 | Unknown function |
| LOC100288152 | Unknown function |
| LOC100419583 | Unknown function |
| LOC100507144 | Unknown function |
| LOC101926911 | Unknown function |
| LOC283575 | Unknown function |
| LPAR5 | Signalling protein |
| LPIN1 | Triglyceride synthesis |
| LSP1 | Actin binding protein |
| LTBR | Cell signalling |
| LYG2 | Lysozyme |
| MALAT1 | long non-coding RNA |
| MAPRE3 | Cell Division |
| MB21D1 | Signalling protein |
| MEFV | Immunomodulator |
| MIR142 | microRNA |
| MILR1 | Signalling protein |
| MIR3124 | microRNA |
| MIR3188 | microRNA |
| MIR3196 | microRNA |
| MIR320E | microRNA |
| MIR330 | microRNA |
| MIR3675 | microRNA |
| MIR4316 | microRNA |
| MIR4488 | microRNA |
| MIR4496 | microRNA |
| MIR4513 | microRNA |
| MIR4674 | microRNA |
| MIR4707 | microRNA |
| MIR4772 | microRNA |
| MIR6088 | microRNA |
| MIR6129 | microRNA |
| MIR6780A | microRNA |
| MIR6797 | microRNA |
| MIR6803 | microRNA |
| MIR6810 | microRNA |
| MIR6842 | microRNA |
| MIR7155 | microRNA |
| MOSPD3 | Signalling protein |
| MPZL1 | Signalling protein |
| MTHFR | Enzyme |
| MTRNR2L3 | Unknown function |
| MYL12A | Myosin light chain |
| NEK6 | Signalling protein |
| NFKBID | NFKB binding protein |
| NTRK1 | Signalling protein |
| OPA3 | Mitochondrial membrane protein |
| OR2V1 | Signalling protein |
| PCID2 | RNA export |
| PCOLCE | Collagen metabolism |
| PCOLCE-AS1 | ncRNA |
| PHKG2 | Signalling protein |
| PIGV | Signalling protein |
| PIM1 | Signalling protein |
| PLEKHG2 | Signalling protein |
| PLEKHG4 | Signalling protein |
| PLEKHM3 | Signalling protein |
| PLK3 | Signalling protein |
| PNKD | Enzyme |
| POLR2E | DNA polymerase II subunit E |
| PP7080 | Unknown function |
| PPM1N | Protein phosphatase |
| PPP1R35 | Protein phosphatase |
| PRUNE | phosphoesterase |
| PTPN23 | Protein phosphatase |
| PTPN6 | Protein phosphatase |
| PTTG1IP | Transcription factor |
| RAB1A | GTPase |
| RABAC1 | GTPase |
| RAE1 | RNA export |
| RAMP2 | Signalling protein |
| RAMP2-AS1 | ncRNA |
| RCSD1 | Actin filament binding |
| RHPN1 | Signalling protein |
| RHPN1-AS1 | ncRNA |
| RILPL2 | Signalling protein |
| RNF144A | Transcription factor |
| RNF144A-AS1 | ncRNA |
| RNU1-1 | Core U1 spliceosome subunit |
| RNU1-2 | Core U1 spliceosome subunit |
| RNU1-27P | Core U1 spliceosome subunit |
| RNU1-28P | Core U1 spliceosome subunit |
| RNU1-3 | Core U1 spliceosome subunit |
| RNU1-4 | Core U1 spliceosome subunit |
| RNU5D-1 | Core U1 spliceosome subunit |
| RNU5F-1 | Core U5 spliceosome subunit |
| RNVU1-18 | Core U1 spliceosome subunit |
| RPS26 | Ribosomal protein |
| RTN2 | Vesicle transport |
| SAA4 | Folate metabolism |
| SCNN1A | ion channel |
| SEPHS2 | Selenophosphate Synthetase |
| SH3BP5 | Signalling protein |
| SH3BP5L | Unknown function |
| SHE | Unknown function |
| SIGLEC8 | Signalling protein |
| SLC27A5 | Solute carrier |
| SLC7A7 | Solute carrier |
| SLFN12L | Unknown function |
| SMARCC2 | Chromatin regulator |
| SNHG12 | snoRNA |
| SNHG15 | snoRNA |
| SNORA16A | snoRNA |
| SNORA17 | snoRNA |
| SNORA44 | snoRNA |
| SNORA9 | snoRNA |
| SNORD10 | snoRNA |
| SNORD12 | snoRNA |
| SNORD12C | snoRNA |
| SNORD9 | snoRNA |
| SNX8 | phosphatidylinositol binding |
| SORBS1 | Signalling protein |
| SORT1 | Protein trafficking |
| SP1 | Transcription factor |
| SPIB | Transcription factor |
| ST6GAL1 | glycosyltransferase |
| STX6 | Protein trafficking |
| TAPBPL | Antigen processing |
| TCOF1 | Ribosome biogenesis |
| TDRD10 | Nucleotide binding |
| TMEM138 | Signalling protein |
| TMEM180 | Signalling protein |
| TMEM265 | Signalling protein |
| TNFAIP8L2 | Innate immunity |
| TNFAIP8L2-SCNM1 | Unknown function |
| TOP1MT | DNA topoisomerase |
| TOP2A | DNA topoisomerase |
| TRIM69 | Transcription factor |
| UBALD2 | Unknown function |
| UBC | ubiquitination |
| UQCC3 | ubiquitination |
| USP3 | ubiquitination |
| USP8 | ubiquitination |
| VASP | Actin binding protein |
| VEGFB | Angiogenesis factor |
| VPS33B | Organelle formation |
| VTRNA1-2 | ncRNA |
| ZDHHC18 | Transcription factor |
| ZFAS1 | ncRNA |
| ZNF131 | Transcription factor |
| ZNF234 | Transcription factor |
| ZNF512B | Transcription factor |
| ZNF688 | Transcription factor |
| ZNF706 | Transcription factor |
| ZNF785 | Transcription factor |

Also interesting was the observation that in 4 cases, a co-ordinate module of coding RNA and its cognate non-coding RNA regulator were both targeted by FOXO1 and ETV6 (AP4B1 and AP4B1-AS1; CD27 and CD27-AS1; PCOLCE and PCOLCE-AS1; RAMP2 and RAMP2-AS1). Gene set enrichment analysis (GSEA) suggested that FOXO1 and ETV6 targeted genes were clustered into pathways involved in senescence processes. The top 4 most associated pathways for targets of both genes were “senescence related secretory phenotype (SASP)” (p=0.0002 for ETV6 and 0.0003 for FOXO1), “Mitotic prophase” (p=0.0004 for ETV6 and 0.0003 for FOXO1), “Cellular senescence” (p=0.0021 for ETV6 and 0.0001 for FOXO1) and “M-Phase” (p=0.0045 for ETV6 and 0.00017 for FOXO1) (see Table 4 below).

TABLE 4
GSEA pathways analysis of FOXO1 and ETV6 target genes

| | | |
|---|---|---|
| Term | P value | Combined score |

ETV6

| | | |
|---|---|---|
| Senescence-Associated | 0.0002 | 17.09 |
| Secretory Phenotype (SASP) | | |
| Mitotic prophase | 0.0004 | 16.13 |
| Cellular Senescence | 0.0021 | 13.75 |
| M phase | 0.0045 | 12.73 |
| RMTs methylate histone arginines | 0.0024 | 12.18 |
| HOX gene activation in differentiation | 0.0027 | 11.49 |
| HOX gene activation in hindbrain | 0.0027 | 11.44 |
| RNA Polymerase I Promoter Opening | 0.0034 | 10.95 |
| Transcriptional regulation by small RNAs | 0.0040 | 10.84 |
| Cell Cycle, Mitotic | 0.0091 | 10.76 |

FOXO1

| | | |
|---|---|---|
| Mitotic Prophase | 0.0003 | 16.67 |
| Senescence-Associated | 0.0003 | 15.96 |
| Secretory Phenotype (SASP) | | |
| Cellular Senescence_{—} | 0.0001 | 15.94 |
| M Phase_{—} | 0.0017 | 15.46 |
| Cell Cycle, Mitotic | 0.0044 | 13.02 |
| Transcriptional regulation by small RNAs | 0.0018 | 12.66 |
| RMTs methylate histone arginines | 0.0021 | 12.26 |
| Transcription-Coupled Nucleotide | 0.0023 | 12.24 |
| Excision Repair (TC-NER) | | |
| Cellular responses to stress_{—} | 0.0054 | 11.78 |
| Formation of TC-NER Pre-Incision Complex | 0.0039 | 10.70 |

### Discussion

Receptor tyrosine kinases integrate multiple signals from the interior and exterior of cells, and communicate this information to the cellular regulatory machinery. Our data suggest that proteins in ERK and AKT signalling pathways show higher levels of phosphorylation in late passage cells, and show significant cross- and auto-regulation. We propose that a major downstream consequence of this may be dysregulation of splicing factor expression in late passage cells, mediated primarily through altered activity and cross reactivity of the FOXO1 and ETV6 transcription factors, and that these changes are linked to senescence phenotypes in this system. Low dose chemical inhibition of either ERK or AKT signalling, or reduction of the expression of FOXO1 or ETV6 genes in late passage human primary fibroblasts resulted in restoration of splicing factor expression to levels consistent with those seen in younger passage cells, reversal of senescence and re-entry to cell cycle for a proportion of the cells tested.

ERK and AKT signalling pathways can be activated by classical ageing stimuli such as DNA damage, dysregulated nutrient signalling and the chronic inflammation of ageing (Fontana et al. 2012; Lin et al. 2013). The NF-kβ pathway, a major contributor to the senescence-associated secretory phenotype (SASP), is also known to be activated by both ERK and AKT signalling (Lin et al. 2012), raising the possibility of a vicious cycle of positive feedback. Dysregulation of normal splicing processes is a key feature of many age-related diseases such as Alzheimer's disease, Parkinson's disease and cancer (Latorre & Harries 2017). Altered splicing regulation is itself associated with ageing in human populations (Harries et al. 2011), with cellular senescence in in vitro models (Holly et al. 2013) and with longevity in mouse models (Lee et al. 2016). Several studies have suggested that splicing regulation may be on the causal pathway to ageing, since targeted disruption of specific splicing factors is able to moderate lifespan in invertebrate models (Heintz et al. 2016). The inventors recent work suggests that features of cellular senescence can be reversed by small molecule restoration of splicing factor levels (Latorre et al. 2017). Recent thinking suggests that changes in the decision-making processes surrounding precisely which isoforms are expressed from genes may contribute directly to ageing and age-related phenotypes (Deschenes & Chabot 2017).

Altered cellular signalling is a key hallmark of ageing. The action of pathways such as mTOR and IGF-1 signalling are well known and well defined (Cohen & Dillin 2008). ERK and AKT signalling have both previously been implicated in ageing and senescence phenotypes (Demidenko et al. 2009; Chappell et al. 2011), and modification of these pathways is also associated with lifespan extension and ageing phenotypes in animal models (Slack et al. 2015). Both ERK and AKT have been previously associated with regulation of splicing factor activity (Shin & Manley 2004; Tarn 2007), and AKT is known to have a role in the regulation of splicing factor genes at the level of kinase activation (Blaustein et al. 2005). However, study of the precise underlying mechanisms by which these pathways may regulate splicing factor expression and influence and ageing phenotypes is fraught with difficulty since they demonstrate definite tissue, dose and context effects, and there is also significant crosstalk between them (Rhim et al. 2016), as the inventors have demonstrated. This implies that results may not be consistent with different doses of inhibitor, and effects seen in one cell type may not necessarily hold true in another. This was evident in our data, where we noted definite effects of dose. At low dose, we saw increased expression of some splicing factors, rescue of senescence phenotypes and re-entry into cell cycle, whereas at higher dose, decreases in splicing factor expression and no increase in proliferative index were noted. This suggests that in case of high dose activation, other feedback loops within AKT or ERK signalling pathways that lead to inhibition of splicing factor expression and proliferation may be activated. Again, this may arise from feedback loops within AKT or ERK signalling pathways leading to inhibition of splicing factor expression and proliferation which may be activated at higher concentrations. This is in itself not unusual, since antagonistic effects in terms of dose or tissue response are not uncommon in cellular signalling pathways (Pardo et al. 2003; Wang et al. 2017). Indeed, our data indicate the presence of a number of cross-regulatory and auto-regulatory feedback loops, which may indicate that splicing factor expression is tightly controlled within fairly narrow expression limits by homeostatic mechanisms, which would be in keeping with their know role in control of proliferation and the combinatorial, dose-responsive nature by which they regulate alternative splicing (Kang et al. 2009; Anczukow et al. 2012; Fu & Ares 2014).

A better understanding of the precise relationship between altered ERK and AKT signalling, splicing factor expression and cellular senescence requires finer molecular dissection. Our data suggest that may of the features of AKT and ERK activation on splicing factor expression and cell senescence phenotypes can be replicated by targeted disruption of two key downstream effectors of ERK or AKT signalling; FOXO1 and ETV6. The FOXO1 and ETV6 genes both encode transcription factors, the closest drosophila homologues of which (Foxo and Aop) have been reported to contribute to the effects of ERK and AKT activation on lifespan in D. melanogaster (Slack et al. 2015). FOXO proteins have a long history of involvement in ageing pathways; they are well-known players in longevity in nematodes, files, and mammals (Salih & Brunet 2008), but to our knowledge have never been linked previously to the regulation of splicing factors.

Targeted knockout of either gene resulted in both increased splicing factor expression and rescue from senescence. Again, the relationship between these genes may not be synergistic, since simultaneous knockdown of both genes results not in an additive or multiplicative effect on splicing factor expression and senescent cell load, but to a complete abrogation of effect. This may partially be explained by a reciprocal regulation of FOXO1 and ETV6 genes at the level of transcription and protein sub-cellular localisation; knockdown of FOXO1 results in increased ETV6 expression and moderation of either signalling pathway by specific inhibitors induces effects on the nuclear localisation of both genes. Several splicing factors have evolutionally-conserved FOXO binding motifs in their promoter regions (Webb et al. 2016), and FOXO1 protein has been reported to co-localise with the nuclear speckles within the cell where splicing occurs (Arai et al. 2015). ETV6 is a member of the ETS family of transcription factors, and is perhaps a less obvious candidate for a longevity gene, although it has a well-known role in control of cellular proliferation and in haematopoietic cancer (Hock & Shimamura 2017). Like FOXO1, ETV6 has similarly not previously been reported as a regulator of splicing factor expression, but other members of the wider ETS family of genes, which have very similar binding sites, have been reported to have such activity (Kajita et al. 2013). Both FOXO1 and ETV6 have been reported to have activity as tumour suppressor genes (Dansen & Burgering 2008; Rasighaemi & Ward 2017), so a role in negatively regulating the expression of genes required for cellular proliferation is not unexpected.

The ChIP analyses indicate that although FOXO1 and ETV6 may directly regulate some splicing factors (HNRNPF, HNRNPL), most regulators of splicing factor expression, but rather act through a series of intermediates, almost 25% of which are non-coding RNAs. Splicing processes are implicated directly, since 9 of the common targets are components of the U1 or U5 spliceosomal complex. There is surprising overlap between the genes regulated by FOXO1 and ETV6, indicating that the regulation may be co-ordinate or competitive. We assume that the regulation may also include the action of other FOXOs and other ETS family members. The role of these genes in mediating senescence processes is also suggested by our GSEA results. FOXO1 and ETV6 targets cluster in pathways fundamental to senescence (“senescence-associated secretory phenotype (SASP)”, “cellular senescence, “M-Phase”, “mitotic cell cycle). Thus our data provide evidence that FOXO1 and ETV6 may coordinately regulate a module of mediator genes that are involved in the regulation of splicing factors and influence their relationship with senescence.

Although our data suggest that ETV6 and FOXO1 may have activity as novel regulators of splicing factor expression, it does not rule out the contribution of other genes in these networks. Indeed, although the overall picture is similar, some splicing factor genes behave differently when challenged with inhibition of the whole pathway compared with specific inactivation of FOXO1 or ETV6 (e.g. SRSF3, SRSF6 for ERK signalling and AKAP17A, LSM2 and LSM14A for AKT signalling). This strongly suggests the presence of other regulators. It is also clear that only a subset of cells are rescued, since the whole cell population does not revert. Even ‘clonal’ cultures of senescent cells are heterogeneous, containing growth-arrested, but non-senescent cells and senescent cells, depending on the degree of paracrine inhibition. The percentage of senescent cells at growth arrest can range from approximately 40% to over 80%, even in different cell lines from the same tissue type.

In conclusion, we present here evidence that activation of ERK and/or AKT signalling by age-related phenomena may lead to a cascade of events culminating in altered activity of FOXO1 and ETV6 transcription factors. We demonstrate here for the first time that ETV6 and FOXO1 represent novel regulators of splicing factor expression, and that age-related alterations in their activity could lead dysregulated splicing and ultimately cellular senescence. Inhibitors of AKT and ERK signalling pathways are already in use as therapeutic agents for cancer, raising the interesting possibility that genes in these pathways may represent targets for early intervention for healthier ageing in the future.

### EXPERIMENTAL PROCEDURES

### Culture of Human Primary Fibroblasts

Normal human dermal fibroblasts derived from neonatal foreskin from a single donor (NHDF; Promocell, Heidelburg, Germany) were cultured in fibroblast growth medium (C-23020, Promocell, Heidelburg, Germany) supplemented with 2% FBS, growth factors (recombinant fibroblast growth factor and recombinant human insulin), 100U/ml penicillin, and 100 ug/ml streptomycin in a seeding density of 6×10³ cell/cm². Early passage cells at population doubling (PD)=25 (85% growth fraction and 7% senescent fraction) or late passage cells at PD=63 (33% growth fraction and 58% senescent fraction) were maintained in culture for 10 days prior to subsequent experiments. Inhibitors of MEK/ERK (trametinib) and AKT (SH-6) were added at 1.1 μM or 10 μM for 24h, based on previous work in the literature (Krech et al. 2010; Slack et al. 2015). Vehicle-only (DMSO) controls were included for each experiment. Where serum starvation was required to differentiate senescence rescue from altered proliferation kinetics, cells were maintained in DMEM (Sigma Aldrich, Dorset, UK) supplemented with 0.1% of serum and 1% penicillin and streptomycin in the absence of fibroblast-specific supplement, for 24 h prior to treatment.

### Assessment of Phosphorylation Changes in Key Signalling Pathways During Cellular Senescence

Early and late passage human primary fibroblasts cells were plated in 25 cm² flasks at a density of 6×10³ cells/cm² and grown for 10 days. Phosphorylation status for proteins in key signalling pathways thought to be activated in senescence was then assessed in cell lysates from early passage (PD=25) or late passage (PD=63) primary human fibroblast cells using the human MAPK phosphorylation antibody array (ab211061, Abcam, Bristol, UK) according to the manufacturer's instructions. Phosphorylation sites tested were: AKT (pS473), CREB (p5133), ERK1 (pT202/Y204), ERK2 (pT185/Y187), GSK3a (pS21), GSKb (pS9), HSP27 (pS82), JNK (pT183), MEK (pS217/221), MKK3 (p5189), MKK6 (pS207), MSK2 (p5360), mTOR (pS2448), p38 (pT180/Y182), p53 (pS15), P70S6K (pT421/5424), RSK1 (p5380). In brief, membranes were blocked with blocking buffer for 30 min at room temperature and incubated with 1 ml of cell lysate overnight at 4° C. After washing, detection antibody cocktail was added and incubated for 2 hours, followed by a 2-hours incubation with HRP-anti-rabbit IgG at room temperature. Membranes were incubated with detection buffer and results were documented on a chemiluminescence imaging system (LI-COR biosciences, Nebraska USA). Signal intensity was quantified using Image Studio software V5.2 (LI-COR biosciences, Nebraska USA). Results were normalised to total cellular protein content and expressed relative to the positive control.

### Assessment of Cellular Senescence

Late passage primary human fibroblasts at PD=63 were seeded in 3 biological replicates of 6×10⁴ cells per well in 6-well plates. Cells were treated with the MEK/ERK inhibitor trametinib or the AKT inhibitor SH-6 at 1 μM and 10 μM, or with a combination of the two inhibitors at 1 μM each for 24 hrs. Cell senescence was then assessed using the biochemical senescence marker SA β-Gal, tested in triplicate using a commercial kit (Sigma Aldrich, UK); according to manufacturer's instructions, with a minimum of 400 cells assessed per replicate. Senescence was also quantified in molecular terms by assessing CDKN2A (p16) gene expression and by changes in cell morphology typical of senescence as in our previous work (Holly et al. 2013). CDKN2A (p16) was measured by qRTPCR relative to GUSB, PP/A and GADPH endogenous control genes, on the QuantStudio 12K Flex platform (Applied Biosystems, Foster City, USA). PCR reactions contained 2.5 μl TaqMan Universal Mastermix (no AMPerase) (Applied Biosystems, Foster City, USA), 0.25 μM probe and 0.5 μl cDNA reverse transcribed as above in a total volume of 5 μl. PCR conditions were a single cycle of 95° C. for 10 minutes followed by 40 cycles of 95° C. for 15 seconds and 60° C. for 1 minute. Quantitative RTPCR assay accession numbers for p16 and p21 are given in table 5 below.

TABLE 5
Sequences of qRTPCR assays, siRNAs and
morpholinos used in this study

| | | Transcript |
|---|---|---|
| Gene name | Probe | accession |
| CDKN2A | N/A | Hs00923894_m1 |
| p21 (CDKN1A) | N/A | Hs00355782_m1 |
| FOXO1 | N/A | Hs00231106_m1 |
| ETV6 | N/A | Hs00231101_m1 |
| Splicing | N/A | |
| factors | | |
| AKAP17A | N/A | Hs00946624_m1 |
| HNRNPA0 | N/A | Hs00246543_s1 |
| HNRNPA1 | N/A | Hs01656228_s1 |
| HNRNPA2B1 | N/A | Hs00242600_m1 |
| HNRNPH3 | N/A | Hs01032113_g1 |
| HNRNPK | N/A | Hs00829140_s1 |
| HNRNPM | N/A | Hs00246018_m1 |
| HNRNPUL2 | N/A | Hs00859848_m1 |
| IMP3 | N/A | Hs00251000_s1 |
| LSM14A | N/A | Hs00385941_m1 |
| LSM2 | N/A | Hs01061967_g1 |
| PNISR | N/A | Hs00369090_m1 |
| HNRNPD | N/A | Hs01086914_g1 |
| SF3B1 | N/A | Hs00202782_m1 |
| SRSF1 | N/A | Hs00199471_m1 |
| SRSF2 | N/A | Hs00427515_g1 |
| SRSF3 | N/A | Hs00751507_s1 |
| SRSF6 | N/A | Hs00607200_g1 |
| TRA2B | N/A | Hs00907493_m1 |
| SRSF7 | N/A | Hs00196708_m1 |
| siRNA | | |
| FOXO1 | ID: s5257 | NM_002015.3 |
| ETV6 | ID: s533885 | NM_001987.4 |
| morpholino | | |
| FOXO1 | TCCCCCAGCCGCAGGAGAGCCAAGA | |
| | (SEQ ID No. 1) | |
| ETV6 | CATGTCTCACAGCGAGAGAGATCAG | |
| | (SEQ ID No. 2) | |

### Quantification of Treatment-Related Changes to SASP Protein Expression

Late passage primary human fibroblast cells at PD=63 were seeded at 6×10⁴ cells per well in a 6 well plate, cultured for 10 days and then treated with at 1 μM or 10 μM trametinib (MEK/ERK inhibitor) or SH-6 (AKT inhibitor) for 24 hours. Cell supernatants were then harvested and stored at −80° C. Levels of GMCSF, IFNγ, IL1β, IL2, IL6, IL8, IL10, IL-12p70, and TNFα SASP components were measured in cell supernatants from treated and vehicle-only control cells using the K15007B MesoScale Discovery multiplex ELISA immunoassay (MSD, Rockville, USA) in 4 biological replicates. Proteins were quantified relative to a standard curve using a Sector Imager SI-6000 according to the manufacturer's instructions.

Assessment of Splicing Factor Expression in Late Passage Primary Human Fibroblasts Treated with ERK or AKT Inhibition

Late passage primary human fibroblasts at PD=63 were seeded at 6×10⁴ cells per well as 3 biological replicates in 6-well plates, allowed to grow for 10 days and then treated with the ERK inhibitor trametinib or the AKT inhibitor SH-6 at 1 μM or 10 μM for 24 hrs. Vehicle (DMSO) only controls were also included under the same growth conditions. The expression levels of 20 splicing factor transcripts previously associated with age, replicative senescence or lifespan in our previous work (Harries et al. 2011; Holly et al. 2013; Lee et al. 2016) were then assessed by qRTPCR. Accession numbers for splicing factor assays are given in Table 5. RNA was extracted using TRI Reagent® (Life Technologies, Foster City USA) according to the manufacturer's instructions. Total RNA (100 ng) was reverse transcribed in 20 μl reactions using the Superscript III VILO kit (Life Technologies). Transcript expression was then quantified in duplicate for each biological replicate using TaqMan Low Density Array (TLDA) on QuantStudio 12K Flex (Applied Biosystems, Foster City, USA). Cycling conditions were 1 cycle each of 50° C. for 2 min, 94.5° C. for 10 min and then 40 cycles of 97° C. for 30 s and 57.9° C. for 1 min. The reaction mixes included 50 μl TaqMan Fast Universal PCR Mastermix (Life Technologies), 30 μl dH₂O and 20 μl cDNA template. 100 μl reaction mixture was dispensed into the TLDA card chamber and centrifuged twice for 1 min at 1000 rpm. Transcript expression was assessed by the Comparative Ct approach, relative to the IDH3B, GUSB and PP/A endogenous control genes and normalised to their expression in RNA from untreated late passage cells.

### Assessment of Cell Proliferation

The proliferative capacity of treated cells was assessed by Ki67 and BrdU staining and by cell counts. Late passage primary human fibroblasts at PD=63 were seeded at 1×10⁴ cells per well in 3 biological replicates in 24-well plates, allowed to grow for 10 days and then assayed for cell proliferation by ki67 staining. For assessment of cells in S phase, late passage primary human fibroblasts at PD=63 were seeded at 1×104 cells per coverslip as 3 biological replicates in 24-well plates, allowed to grow for 10 days and then were incubated with 5-Bromo-2′-deoxy-uridine (BrdU) for 24 hrs. BrdU incorporation was determined by the 5-Bromo-2′-deoxy-uridine labeling and detection kit I following instructions of the manufacturer (Roche Molecular Biochemicals). BrdU positive cells were visualized and counted by fluorescence microscopy. Cell counts were carried out manually in 3 biological replicates in treated and vehicle-only cultures following trypsinisation and suspension of cells and are presented as mean (+/−SEM). Following treatment, cells were fixed for 10 min with 4% PFA and permeabilized with 0.025% Triton and 10% serum in PBS for 1 hour. Ki-67 staining was carried out by rabbit monoclonal antibody (ab16667, Abcam, UK) at a 1:400 dilution and samples were incubated overnight at 4° C., followed by FITC-conjugated secondary goat anti-rabbit antibody (1:400) for 1 hour, and nuclei were counterstained with DAPI. Coverslips were mounted on slides in DAKO fluorescence mounting medium (S3023; Dako, Santa Clara, USA). The proliferation index was determined by counting the percentage of Ki67 positive cells from at least 400 nuclei from each biological replicate at 400× magnification under a Leica D4000 fluorescence microscope.

### Assessment of Apoptosis Using TUNEL Assay

Terminal DNA breakpoints in situ 3-hydroxy end labeling (TUNEL) assay was to quantify levels of apoptosis in NHDF cells. Late passage primary human fibroblasts at PD=63 were seeded at 1×10⁴ cells per well as 3 biological replicates in 24-well plates, allowed to grow for 10 days and a TUNEL assay was performed with Click-iT® TUNEL Alexa Fluor® 488 Imaging Assay kit (Thermofisher, UK) following the manufacturer's instructions. Negative (vehicle only) and positive (DNase1) controls were also performed. The apoptotic index was determined by counting the percentage of positive cells from at least 400 nuclei from each biological replicate at 400× magnification.

### ETV6 and FOXO1 Gene Knockdown

Previous work in invertebrate systems has identified the Aop and Foxo genes, involved in ERK and AKT signalling respectively, as determinants of lifespan in D. melanogaster (Slack et al. 2015). The closest human homologues of these genes are FOXO1 (Kramer et al. 2003) and ETV6/TEL (Jousset et al. 1997). We assessed the effect of knockdown of FOXO1 or ETV6 gene expression on cellular senescence and splicing factor expression in late passage primary human fibroblasts. Late passage cells at PD=63 were seeded at 6×10⁴ cells per well into 6-well plates were cultured for 10 days. Antisense oligonucleotides (morpholinos, MOs) were designed to the 5′ untranslated region of the FOXO1 or ETV6 genes, in the vicinity of the initiation codon (Gene Tools LLC, Philomath, USA). Morpholino oligonucleotides (10 μM) were introduced into the cells by endo-porter delivery according to the manufacturer's instructions. A fluorescein-conjugated scrambled negative control morpholino was also included as a negative control and to monitor delivery of constructs. Transfection efficiency was assessed by microscopy. Splicing factor expression and cellular senescence were then determined as described above. Results were confirmed by another method of gene knockdown; siRNA. For this work, late passage primary human fibroblasts at PD=63 were seeded in 3 biological replicates of 6×10⁴ cells per well in 6-well plates and transfections were carried out using 15 nM FOXO1, ETV6 siRNA or 15 nM control siRNA (Themofisher) using Lipofectamine RNAiMAX reagent (Invitrogen, Paisley, UK) for 48 hr. siRNA and morpholino sequences are given in Table 5.

### Assessment of FOXO1 and ETV6 Subcellular Localization

The subcellular localization of ETV6 and FOXO1 proteins were assessed by immunofluorescence. Late passage primary human fibroblasts at PD=63 were seeded at 1×104 cells per coverslip as 3 biological replicates in 24-well plates, allowed to grow for 10 days and then assayed for subcellular localisation. Following treatment with 1.1 μM trametinib or SH-6, cells were fixed for 10 min with 4% PFA and permeabilized with 0.025% Triton and 10% serum in PBS for 1 hour. Anti-rabbit ETV6 (ab64909) and anti-rabbit FOXO1 from (Abcam, UK) at a 1:1000 and 1:100 dilution respectively and samples were incubated overnight at 4° C., followed by FITC-conjugated secondary goat anti-rabbit antibody (1:400) for 1 hour, and nuclei were counterstained with DAPI. Coverslips were mounted on slides in DAKO fluorescence mounting medium (S3023; Dako, Santa Clara, USA). The nuclear localization was determined by counting the percentage of nuclear staining from at least 50 cells from each biological replicate at 400× magnification under a Leica D4000 fluorescence microscope.

### Assessment of Telomere Length

DNA was extracted from 2×10⁵ late passage primary human fibroblasts at PD=63 which had been plated in 3 biological replicates and then treated with 1.1 μM of either the ERK inhibitor trametinib or the AKT inhibitor SH-6 for 24 hrs, using the PureLink® Genomic DNA Mini Kit (Invitrogen™/Thermo Fisher, Mass., USA) according to the manufacturer's instructions. DNA quality and concentration was checked by Nanodrop spectrophotometry (NanoDrop/Thermo Fisher, Mass., USA). Relative telomere length was determined using a modified qPCR protocol (O'Callaghan & Fenech 2011). PCR reactions contained 1 μl EvaGreen (Solis Biodyne, Tartu, Estonia), 2 μM each primer and 25 ng DNA in a total volume of 5 μI in a 384 well plate. The quantitative real time polymerase chain reaction telomere assay was run on the StepOne Plus, cycling conditions were: a single cycle of 95° C. for 15 minutes followed by 45 cycles of 95° C. for 10 seconds, 60° C. for 30 seconds and 72° C. for 1 minute. The average relative telomere length was calculated as the ratio of telomere repeat copy number to a single copy number gene (3684) and normalised to telomere length in untreated cells.

### Statistical Analysis

Unless otherwise indicated, differences between treated and vehicle-only control cultures were assessed for statistical significance by two way ANOVA analysis. Statistical analysis was carried out with the computer-assisted Prism GraphPad Program (Prism version 5.00, GraphPad Software, San Diego, Calif.).

### Chromatin Immunoprecipitation (ChIP) and Gene Set Enrichment Analysis

Input data for this work was 4 publically available human ChIP datasets for ETV6 and 3 for FOXO1. ETV6 datasets comprised 2 sets derived from K562 cells (GSE91511 and GSE95877), and 2 datasets derived from GM12878 cells (GSE91904 and GSE96274). FOXO1 datasets comprised datasets from human endometrial stromal cells (GSE69542), human pre-leukaemia B cells (GSE80773) and normal human B cells (GSE68349). These datasets were imported into Cistrome Project software (www.cistrome.org) for identification of FOXO1 and ETV6 target genes using the BETA (Binding and Expression Target Analysis) minus application using default parameters. This software detects transcription factor binding sites in input data based on regulatory potential score, following filtering of peaks with less than 5 fold signal to background ratio. GSEA pathway analysis was then carried out using the Enrichr program using the 2016 reactome interface.

The forgoing embodiments are not intended to limit the scope of the protection afforded by the claims, but rather to describe examples of how the invention may be put into practice.

### REFERENCES

Anczukow O, Rosenberg A Z, Akerman M, Das S, Zhan L, Karni R, Muthuswamy S K, Krainer A R (2012). The splicing factor SRSF1 regulates apoptosis and proliferation to promote mammary epithelial cell transformation. Nat Struct Mol Biol. 19, 220-228.
Arai T, Kano F, Murata M (2015). Translocation of forkhead box 01 to the nuclear periphery induces histone modifications that regulate transcriptional repression of PCK1 in HepG2 cells. Genes Cells. 20, 340-357.
Baar M, Brandt R, Putavet D, Klein J, Derks K, Bourgeois B, Stryeck S, Rijksen Y, van Willigenburg H, Feijtel D, van der pluijm I, Essers J, Van Cappellen W A, van I Jcksen W, Houtsmuller A B, Pothof J, De Bruin R W, Madi T, Hoeijmakers J G, Campisi J, de Keizer P (2017). Targeted Apoptosis of Senescent Cells Restores Tissue Homeostasis in Response to Chemotoxicity and Aging. Cell 169, 133 to 147.
Baker D J, Childs B G, Durik M, Wijers M E, Sieben C J, Zhong J, Saltness R A, Jeganathan K B, Verzosa G C, Pezeshki A, Khazaie K, Miller J D, van Deursen J M (2016). Naturally occurring p16(Ink4a)-positive cells shorten healthy lifespan. Nature. 530, 184-189.
Blaustein M, Pelisch F, Tanos T, Munoz M J, Wengier D, Quadrana L, Sanford J R, Muschietti J P, Kornblihtt A R, Caceres J F, Coso O A, Srebrow A (2005). Concerted regulation of nuclear and cytoplasmic activities of S R proteins by AKT. Nat Struct Mol Biol. 12, 1037-1044.
Bullock N, Oltean S (2017). The many faces of SRPK1. J Pathol. 241, 437-440.
Chappell W H, Steelman L S, Long J M, Kempf R C, Abrams S L, Franklin R A, Basecke J, Stivala F, Donia M, Fagone P, Malaponte G, Mazzarino M C, Nicoletti F, Libra M, Maksimovic-Ivanic D, Mijatovic S, Montalto G, Cervello M, Laidler P, Milella M, Tafuri A, Bonati A, Evangelisti C, Cocco L, Martelli A M, McCubrey J A (2011). Ras/Raf/MEK/ERK and PI3K/PTEN/Akt/mTOR inhibitors: rationale and importance to inhibiting these pathways in human health. Oncotarget. 2, 135-164.
Cohen E, Dillin A (2008). The insulin paradox: aging, proteotoxicity and neurodegeneration. Nature reviews. Neuroscience. 9, 759-767.
Dansen T B, Burgering B M (2008). Unravelling the tumor-suppressive functions of FOXO proteins. Trends Cell Biol. 18, 421-429.
de Magalhaes J P (2004). From cells to ageing: a review of models and mechanisms of cellular senescence and their impact on human ageing. Exp Cell Res. 300, 1-10.
Demidenko Z N, Shtutman M, Blagosklonny M V (2009). Pharmacologic inhibition of MEK and P1-3K converges on the mTOR/S6 pathway to decelerate cellular senescence. Cell cycle. 8, 1896-1900.
Deschenes M, Chabot B (2017). The emerging role of alternative splicing in senescence and aging. Aging Cell.
Faragher R G, McArdle A, Willows A, Ostler E L (2017). Senescence in the aging process. F1000Res. 6, 1219.
Fontana L, Vinciguerra M, Longo V D (2012). Growth factors, nutrient signaling, and cardiovascular aging. Circ Res. 110, 1139-1150.
Fu X D, Ares M, Jr. (2014). Context-dependent control of alternative splicing by RNA-binding proteins. Nat Rev Genet. 15, 689-701.
Harries L W, Hernandez D, Henley W, Wood A R, Holly A C, Bradley-Smith R M, Yaghootkar H, Dutta A, Murray A, Frayling T M, Guralnik J M, Bandinelli S, Singleton A, Ferrucci L, Melzer D (2011). Human aging is characterized by focused changes in gene expression and deregulation of alternative splicing. Aging Cell. 10, 868-878.
Heintz C, Doktor T K, Lanjuin A, Escoubas C C, Zhang Y, Weir H J, Dutta S, Silva-Garcia C G, Bruun G H, Morantte I, Hoxhaj G, Manning B D, Andresen B S, Mair W B (2016). Splicing factor 1 modulates dietary restriction and TORC1 pathway longevity in C. elegans. Nature. 541, 102-106.
Hock H, Shimamura A (2017). ETV6 in hematopoiesis and leukemia predisposition. Semin Hematol. 54, 98-104.
Holly A C, Melzer D, Pilling L C, Fellows A C, Tanaka T, Ferrucci L, Harries L W (2013). Changes in splicing factor expression are associated with advancing age in man. Mechanisms of ageing and development. 134, 356-366.
Jousset C, Carron C, Boureux A, Quang C T, Oury C, Dusanter-Fourt I, Charon M, Levin J, Bernard O, Ghysdael J (1997). A domain of TEL conserved in a subset of ETS proteins defines a specific oligomerization interface essential to the mitogenic properties of the TEL-PDGFR beta oncoprotein. EMBO J. 16, 69-82.
Kajita K, Kuwano Y, Kitamura N, Satake Y, Nishida K, Kurokawa K, Akaike Y, Honda M, Masuda K, Rokutan K (2013). Ets1 and heat shock factor 1 regulate transcription of the Transformer 2beta gene in human colon cancer cells. J Gastroenterol. 48, 1222-1233.
Kang X, Chen W, Kim R H, Kang M K, Park N H (2009). Regulation of the hTERT promoter activity by MSH2, the hnRNPs K and D, and GRHL2 in human oral squamous cell carcinoma cells. Oncogene. 28, 565-574.
Kourtis N, Tavernarakis N (2011). Cellular stress response pathways and ageing: intricate molecular relationships. EMBO J. 30, 2520-2531.
Kramer J M, Davidge J T, Lockyer J M, Staveley B E (2003). Expression of Drosophila FOXO regulates growth and can phenocopy starvation. BMC Dev Biol. 3, 5.
Krech T, Thiede M, Hilgenberg E, Schafer R, Jurchott K (2010). Characterization of AKT independent effects of the synthetic AKT inhibitors SH-5 and SH-6 using an integrated approach combining transcriptomic profiling and signaling pathway perturbations. BMC Cancer. 10, 287.
Lareau L F, Brenner S E (2015). Regulation of splicing factors by alternative splicing and NMD is conserved between kingdoms yet evolutionarily flexible. Mol Biol Evol. 32, 1072-1079.
Latorre E, Birar V C, Sheerin A N, Jeynes J C C, Hooper A, Dawe H R, Melzer D, Cox L S, Faragher R G A, Ostler E L, Harries L W (2017). Small molecule modulation of splicing factor expression is associated with rescue from cellular senescence. BMC Cell Biol. 18, 31.
Latorre E, Harries L W (2017). Splicing regulatory factors, ageing and age-related disease. Ageing Res Rev. 36, 165-170.
Lee B P, Pilling L C, Emond F, Flurkey K, Harrison D E, Yuan R, Peters L L, Kuschel G, Ferrucci L, Melzer D, Harries L W (2016). Changes in the expression of splicing factor transcripts and variations in alternative splicing are associated with lifespan in mice and humans. Aging Cell. 15, 903-913.
Lin G, Tang Z, Ye Y B, Chen Q (2012). NF-kappaB activity is downregulated by KRAS knockdown in SW620 cells via the RAS-ERK-IkappaBalpha pathway. Oncol Rep. 27, 1527-1534.
Lin X, Yan J, Tang D (2013). ERK kinases modulate the activation of PI3 kinase related kinases (PIKKs) in DNA damage response. Histol Histopathol. 28, 1547-1554.
O'Callaghan N J, Fenech M (2011). A quantitative PCR method for measuring absolute telomere length. Biol Proced Online. 13, 3.
Pardo R, Andreolotti A G, Ramos B, Picatoste F, Claro E (2003). Opposed effects of lithium on the MEK-ERK pathway in neural cells: inhibition in astrocytes and stimulation in neurons by GSK3 independent mechanisms. J Neurochem. 87, 417-426.
Pise-Masison C A, Radonovich M, Sakaguchi K, Appella E, Brady J N (1998). Phosphorylation of p53: a novel pathway for p53 inactivation in human T-cell lymphotropic virus type 1-transformed cells. J Virol. 72, 6348-6355.
Rasighaemi P, Ward A C (2017). ETV6 and ETV7: Siblings in hematopoiesis and its disruption in disease. Crit Rev Oncol Hematol. 116, 106-115.
Rhim J H, Luo X, Gao D, Xu X, Zhou T, Li F, Wang P, Wong S T, Xia X (2016). Cell type-dependent Erk-Akt pathway crosstalk regulates the proliferation of fetal neural progenitor cells. Sci Rep. 6, 26547.
Salama R, Sadaie M, Hoare M, Narita M (2014). Cellular senescence and its effector programs. Genes Dev. 28, 99-114.
Salih D A, Brunet A (2008). FoxO transcription factors in the maintenance of cellular homeostasis during aging. Current opinion in cell biology. 20, 126-136.
Salminen A, Kauppinen A, Kaarniranta K (2012). Emerging role of NF-kappaB signaling in the induction of senescence-associated secretory phenotype (SASP). Cell Signal. 24, 835-845.
Shin C, Manley J L (2004). Cell signalling and the control of pre-mRNA splicing. Nat Rev Mol Cell Biol. 5, 727-738.
Slack C, Alic N, Foley A, Cabecinha M, Hoddinott M P, Partridge L (2015). The Ras-Erk-ETS-Signaling Pathway Is a Drug Target for Longevity. Cell. 162, 72-83.
Suh Y, Atzmon G, Cho M O, Hwang D, Liu B, Leahy D J, Barzilai N, Cohen P (2008). Functionally significant insulin-like growth factor I receptor mutations in centenarians. Proceedings of the National Academy of Sciences of the United States of America. 105, 3438-3442.
Tarn W Y (2007). Cellular signals modulate alternative splicing. J Biomed Sci. 14, 517-522.
van Deursen J M (2014). The role of senescent cells in ageing. Nature. 509, 439-446.
van Heemst D, Beekman M, Mooijaart S P, Heijmans B T, Brandt B W, Zwaan B J, Slagboom P E, Westendorp R G (2005). Reduced insulin/IGF-1 signalling and human longevity. Aging Cell. 4, 79-85.
Wang Q, Chen X, Hay N (2017). Akt as a target for cancer therapy: more is not always better (lessons from studies in mice). Br. Cancer. 117, 159-163.
Webb A E, Kundaje A, Brunet A (2016). Characterization of the direct targets of FOXO transcription factors throughout evolution. Aging Cell. 15, 673-685.

1. A composition comprising one or more intermediate non-coding RNA regulators which modulate the expression of ETV6 for use in the prevention, management, amelioration or treatment of an age-related disease or condition or cancer.

2. The composition of claim 1, wherein the age-related disease or condition or cancer involves dysregulation of splicing factor expression and/or dysregulation of cellular senescence.

3. A composition comprising one or more intermediate non-coding RNA regulators which modulate the expression of FOXO1 for use in the prevention, management, amelioration or treatment of an age-related disease or condition or cancer, wherein the age-related disease or condition or cancer involves dysregulation of splicing factor expression and/or dysregulation of cellular senescence.

4. The composition of any one of claims 1-3, wherein the age-related disease or condition is selected from Alzheimer's disease, cardiovascular disease, hypertension, osteoporosis, type 2 diabetes, cancer, Parkinson's disease, cognitive dysfunction, frailty or progeroid syndromes.

5. The composition of any one of claims 1-4, wherein the composition comprises two or more of the intermediate non-coding RNA regulators.

6. The composition of any one of claims 1-5, wherein the intermediate non-coding RNA regulators comprise miRNAs, miRNA mimics or antagomiRs.

7. The composition of claim 6, wherein the intermediate non-coding RNA regulators comprises two or more miRNAs, miRNA mimics or antagomiRs capable of binding to two or more different complementary and antisense miRNA sequences.

8. The composition of any one of claims 5-7, wherein the intermediate non-coding RNA regulators are selected from one or more of the following: MIR142; MIR3124; MIR3188; MIR3196; MIR320E; MIR330; MIR3675; MIR4316; MIR4488; MIR4496; MIR4513; MIR4674; MIR4707; MIR4772; MIR6088; MIR6129; MIR6780A; MIR6797; MIR6803; MIR6810; MIR6842; or MIR7155.

9. The composition of claim 8, wherein the intermediate non-coding RNA regulators are selected from one or more of the following: MIR3124; MIR3675; MIR4496; MIR6780A; MIR6810; MIR6842; or MIR7155.

10. A method of increasing splicing factor expression, reducing cellular senescence and/or promoting re-entry to the cell cycle in a cell culture comprising administering to the cell culture a composition comprising intermediate non-coding RNA regulators which modulate the expression of FOXO1 and/or ETV6.

11. The method of claim 8, wherein the composition comprises two or more of the intermediate non-coding RNA regulators.

12. The method of claim 8 or claim 9, wherein the intermediate non-coding RNA regulators comprise miRNAs, miRNA mimics or antagomiRs.

13. The method of any one of claims 8-10, wherein the expression modulator of FOXO1 and/or ETV6 comprises two or more miRNAs, miRNA mimics or antagomiRs capable of binding to two or more different complementary and antisense miRNA sequences.

14. Use of a composition comprising one or more intermediate non-coding RNA regulators which modulate the expression of ETV6 for the cosmetic treatment of the effects of ageing.

15. Use according to claim 14, wherein the effects of aging involves dysregulation of splicing factor expression and/or dysregulation of cellular senescence.

16. Use of a composition comprising one or more intermediate non-coding RNA regulators which modulate the expression of FOXO1 for the cosmetic treatment of the effects of ageing, where the effects of aging involves dysregulation of splicing factor expression and/or dysregulation of cellular senescence.

17. Use according to any one of claims 14 to 16, wherein the composition comprises two or more of the intermediate non-coding RNA regulators.

18. Use according to any one of claims 14-17, wherein the intermediate non-coding RNA regulators comprise miRNAs, miRNA mimics or antagomiRs.

19. Use according to claim 18, wherein the expression modulator of FOXO1 or ETV6 comprises two or more miRNAs, miRNA mimics or antagomiRs capable of binding to two or more different complementary and antisense miRNA sequences.

20. Use according to any one of claims 14-19, wherein the intermediate non-coding RNA regulators are selected from one or more of the following: MIR142; MIR3124; MIR3188; MIR3196; MIR320E; MIR330; MIR3675; MIR4316; MIR4488; MIR4496; MIR4513; MIR4674; MIR4707; MIR4772; MIR6088; MIR6129; MIR6780A; MIR6797; MIR6803; MIR6810; MIR6842; or MIR7155.

21. Use according to claim 20, wherein the intermediate non-coding RNA regulators are selected from one or more of the following: MIR3124; MIR3675; MIR4496; MIR6780A; MIR6810; MIR6842; or MIR7155.

22. Use of a composition comprising one or more intermediate non-coding RNA regulators which modulate the expression of FOXO1 and/or ETV6 as a research tool for restoring and/or increasing splicing factor expression; or reducing or reversing cell senescence and/or re-entry to cell cycle.

23. Use of a composition comprising one or more intermediate non-coding RNA regulators which modulate the expression of FOXO1 and/or ETV6 for cell culture.

24. Use according to claim 23, wherein the composition is for increasing viable number of passages in cell culture and/or reduce senescent cell populations.
